SEQUENCE LISTING (31 sequences)

SEQ ID NO: 1 - DNA, 2100 bp, Brassica napus
atgccacgtgtttctgctgaatcccaagaaatctctctcgacggcggctgggagtcaccaacgctcggcgaactgctaaaagatctcgaagacggtcaccggaagaaagactccggcgaagatgcttcggttcatcacgtattggatgtcgcttcccctgaaacaagacctgtgccgtttctcttatccttcaacaatctctgttacgatgtcaggggaaaagccgactcggtcaaaactctactcaacgatgtttccggcggggtttgcgacggcgatgtccttgccgttctcggtgcaagcggagccggtaagtccacgttgatcgacgcactagcgggacgtgtgagtagcttgagaggtacggtaactctaaacggagagaaagttttgaaaagtcaactcctaaaagtgatatcagcatacgtcatgcaagacgatcccttgtttccgatgctcaccgtcaaagaaacactaatgttcgcttcagagtttcgtcttccgagaagcttgtccaagtccaagaaaatggagcgtgttgaagccctaatagacaagctagggctcagaaacgcggcggatacaataataggagacgaaggacaccgtggggtctccggcggagagcggcggcgcgtgtcgatcggtgccgacatcatccacgaccccattgtcttgttcctggacgaacctacttcggggttggactccaccaacgcctttatggtggtgcaagttcttaagcgtatcgctcgtagtggcagtatggtaattatgtcgatacatcaacctagcgctcgtatcatagacttgctcgaccgtcttatcatcttatctcgcggcaagagtgtattcagtggatatccgacaagtcttcctcagttcttgtctgatttcggacatccagtcccggggaaagagaacatcacagagtttgcacttgacctagtccgagagctagaaggatcgaccaaaggaaccgaagagttagtagagttcaacgagaagtggcaacagaaccaatctcctcgagccacgccaatgaccactccttacaaagcattgtctctaaaagaatccatcactacaagtgtttccagaggcaaactagtctccggctcgaccagctccaaccccatctcaatggagacagtaccatacgcaaacacgccattggtcgaggcatatatattatccaaacgttacattaaaaactggtcccgcacccccgagctcattataacacggctcgctacggtcctggtgactggtcttatcttagctactatatattggaggctggacaacactccacgaggtgcacaagagagaatggctttcttttcattcgccatgtccacaatgttctacacctgtgcagacaacctccctgtctttatccatgaacgttacattttcttgagagagacaactcacaatgcatacaggacatactcatacgttatatctcacgttctcgtgtctctgcctcagctactcgctctctccattgcatttgctgttaccacgttctggacagttggtttaagcggtggactagagagcttcttgtattaccgtctcattatctacgcagccttttggtctggttcctctttcgttaccttcatatccggtcttattccgaatgtcatgataagtttcatggtcactatttcctatctttcgtactgtctactgatgggtggattcttcattaaccgtgatcggataccgggttactggatatggtttcattacatctcattgatgaagtatccttatgaagctgtctcgatcaatgagtttgatgacccatctcgatgttttgtaagaggagttcaagtatttgatggtacgcttttcgccaaagtgcctgatgcgataaaggttaagatgtttgatacactgggtaactctttaggaactaagataacggagtccacatgcttgagaacagggcctgacttgcttttgcagcaaggtatatctcagttgagcaaatgggattgcttgtgggttacgtttgcttggggtatcttctttaggatcttgttttacttgtccttgttgtttggaagcaagaataaaaggacgtga

SEQ ID NO: 2 - protein, 699 aa, Brassica napus
MPRVSAESQEISLDGGWESPTLGELLKDLEDGHRKKDSGEDASVHHVLDVASPETRPVPFLLSFNNLCYDVRGKADSVKTLLNDVSGGVCDGDVLAVLGASGAGKSTLIDALAGRVSSLRGTVTLNGEKVLKSQLLKVISAYVMQDDPLFPMLTVKETLMFASEFRLPRSLSKSKKMERVEALIDKLGLRNAADTIIGDEGHRGVSGGERRRVSIGADIIHDPIVLFLDEPTSGLDSTNAFMVVQVLKRIARSGSMVIMSIHQPSARIIDLLDRLIILSRGKSVFSGYPTSLPQFLSDFGHPVPGKENITEFALDLVRELEGSTKGTEELVEFNEKWQQNQSPRATPMTTPYKALSLKESITTSVSRGKLVSGSTSSNPISMETVPYANTPLVEAYILSKRYIKNWSRTPELIITRLATVLVTGLILATIYWRLDNTPRGAQERMAFFSFAMSTMFYTCADNLPVFIHERYIFLRETTHNAYRTYSYVISHVLVSLPQLLALSIAFAVTTFWTVGLSGGLESFLYYRLIIYAAFWSGSSFVTFISGLIPNVMISFMVTISYLSYCLLMGGFFINRDRIPGYWIWFHYISLMKYPYEAVSINEFDDPSRCFVRGVQVFDGTLFAKVPDAIKVKMFDTLGNSLGTKITESTCLRTGPDLLLQQGISQLSKWDCLWVTFAWGIFFRILFYLSLLFGSKNKRT

SEQ ID NO: 3 - DNA, 781 bp, Cauliflower mosaic virus
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 4 - DNA, 360 bp, Solanum tuberosum
ttgattttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatagg

SEQ ID NO: 5 - DNA, 2249 bp, Brassica napus
atgccacgtgtttctgctgaatcccaagaaatatcattcgacggcggcagcgaaccgacgctcggagagctcctgaaagatttcgacggaggtgaccggaagaaaaactccggcgaagatgcttcgactcatcacatacttgatctcacatcccctgaaataagacccgtaccgtttctcttgtccttcaacaacctcagctacgacatcgtacatcgccggcggtttgacttctctcgaggaaagccagcttcagtgaaaactctactcaacgatgtttccggcgaggcttgcgacggagacatcctagccgttctcggagcaagcggagcgggaaagtccacgttgatcgacgcgctagcgggacgcgtgagtagcctgagaggcacggtaactctaaacggagagaagatcttgcaaactcgtttgctgaaagtgatatcagcttacgtcatgcaagacgatcttttgttcccgatgctcaccgtcaaagaaactctaatgttcgcttcagagtttcgtctcccgagaagcttgtccaagtccaagaaaatggagcgtgttcaaaccctaatagaccagttagggctcagaaacgcggcggataccataataggagacgagggacaccgtggagtctctggtggagagcggaggcgcgtgtcgataggaatcgatatcatccacgatcctatcctcttgttccttgatgaacctacgtccgggttggattcaaccaacgcgtttatggtggttcaggtaaggcagccggataaaacattcctcttattatcttcaaaatttttatatagtttaccatatgttttaaaataaaattacttacctaaatggctaaatctcagatccggttcggttttcaggttcttaaacgtatagctaggagtggtagtatcgtaattatgacaatacaccaacctagcgctcgagttcttgacttgcttgatcgtcttatcatcttatctcgcggcaagaatgttttcagcggttctccgacaagtcttcctcagttcttgtctgatttcggacatcctatcccggagaaagagaacataaccgagttcgcacttgacctagttcgtcagcttgaaggatctagtgaaggaaccagagagttggttaaattcaacgaaaagtggcaacaaaaccaatctgctcgagccacgccaatgaccacaccttaccaagccttgtctctaaaagaatccattaccgcaagtgtttctagaggcaaactagtctccggttcaaccagttccaatcccatttccatggactcggtatcttcatacgcaaacccacccttggtcgagaccttcatcttagccaaacggtacatgaaaaactggatccggacacccgagctcttagggacaaggatcgccactgtcatggtcactggtcttctcttagctactatatactggaggcttgacaacactccacgaggtgcacaagagcggatggctttctttgcatttggcatgtccacgatgttctacgtctgtgcagacaacgttccagtttttctccaagaacggttcattttcttgagagagacaacgcgcaacgcatacagaacatcttcgtacgtaatctctcactctcttgtctctctgcctcagctacttgctctctcaattgcatttgctgcgaccacgttctggactgttggtttaagcggtggactagagagcttcctttattactgcctcataatctacgcaggcttttggtctggatcctcttttgtcaccttcgtatccggtttggttccgaatgtcatgataagtttcatgatcactattgcctatctttcctactgtctactcttgggtggattctacattaaccgggatcggataccggtttactggatatggtttcattacatttcattgttgaagtatccctacgaagctgtcttaatcaacgagtttgatgacccatctcgctgttttgttagaggagtccaagtgtttgatggtacgcttttggcgaaagtgcctgatgcgatgaaggttaagctcctcgatacactgagtagctctttaggaacaacgataacggagtccacatgcttgagaacagggcctgacttacttatgcagcaaggtatttctcagttgagcaaatgggattgtttgtggattacgttagcttggggtctcttctttaggatcttgttttacttctccttgctgtttggaagcaagaataaaaggacgtga

SEQ ID NO: 6 - DNA, 2236 bp, Brassica napus
atgccacgtgtttctgctgaatcccaagaaatctcattcgacggcggcaacgaaccgacgctcggagagctcctgaaagatttcgacggaggtgaccggaagaaaaactccggcgaagatgcttcgactcatcacatacttgatctcacttcccctgaaacaagacccgtaccgtttctcttgtccttcaacaacctcagctacgacatcgtacatcgccggcggtttgtcttctctcgaggaaagccagcttcagtgaaacctctactcaacgatgtttccggcgaggcttgcgacggagacatcctagccgttctcggagcaagcggagccggaaagtccacgttgatcgacgcgctagcgggacgcgtgggtagcttgagaggcacggtaactctaaacggagagaagatcttgcaaactcgtttgctgaaagtgatatcagcttacgtcatgcaagacgatcttttgttcccgatgctcaccgtcaaagaaactctaatgttcgcttcagagtttcgtctcccgagaagcttgtccaagtccaagaaaatggagcgtgttcaaaccctaatagacaagttagggcttagaaacgcggcggatacgataataggagacgaaggtcaccgtggagtctccggtggagagcggcggcgcgtgtcgataggaatcgatatcatccacgatcctatcctcttgttccttgatgaacctacatccgggttggattcaaccaatgcgtttatggttgtgcaggtcggatgaaacattcgtcttatcttcaaaattttaaatagttactatatatttcaatttttttaaattaaaattactctccgaaatctcagatccggttctgttttcaggttcttaaacgtatagctaggagtggtagtatcgtaattatgacaatacatcaacctagcgctcgagtccttgacttgcttgatcgtcttatcatcttatctcgcggcgagaatgttttcagcggttctccgacaagtcttcctcagttcttgtctgatttcggacatcctatcccggagaaagagaacataaccgagttcgcactcgacctagtacgacaactcgaagggtccagcgaaggaaccagagagttagttgagttcaacgagaagtggcaacagaaccattctgctcgagccacgccaatgaccacaccttaccaagccttgtctctaaaagaatccattaccgcaagtgtttcgagaggcaagctagtctccggttcaaccagttccgatccaatttccatggactctgtatcttcatacgcaaacccgccactggtcgagacctttatcttagccaaacggtacatgaaaaactggatccggacaccggagctcatagggacacggatcgccactgtcatggtgactggtcttctcttagctactatatactggaggcttgacaacactccgagaggtgcacaagagaggatggctttctttgcatttggtatgtcaacaatgttctacgtctgtgcggacaacgttcctgtttttctccaagaacggttcattttcttgagggagacaacgcgcaacgcatacagaacatcttcgtacgtaatctctcactctcttgtctctctgcctcagctacttgctctctcaattgcatttgctgcgaccacgttctggactgttggtttaagcggtggactagagagcttcctttattactgcctcataatctacgcaggcttttggtctggatcctcttttgtcaccttcgtatccggtttggttccgaatgtcatgataagtttcatgatcactattgcctatctttcctactgtctactcttgggtggattctacattaaccgggatcggataccggtttactggatatggtttcattacatttcattgttgaagtatccctacgaagctgtcttaatcaacgagtttgatgacccatctcgctgttttgttagaggagtccaagtgtttgatggtacgcttttggcgaaagtgcctgatgcgatgaaggttaagctcctcgatacactgagtagctctttaggaacaacgataacggagtccacatgcttgagaacagggcctgacttacttatgcagcaaggtatttctcagttgagcaaatgggattgtttgtggattacgttagcttggggtctcttctttaggatcttgttttacttctccttgctgtttggaagcaagaataaaagacgtga

SEQ ID NO: 7 - DNA, 2344 bp, Solanum tuberosum
atgtcaaggatagtagcggaaaatatgttacaagggggagaaaatgtacaattttatgatcaaagagtacaacaagcaatggagatgtcacaagccagcgcgtactcttcacccaccctaggccaaatgctaaagcgcgtgggagacgtgagaaaagaagtcaccggcgacgaaactccggtgcaccggattctcgatatgagtgatactcaaagcatatcatctcactctcttccttttgtactctccttcaacaacctcacctacagcgtaaaagttcgccggaaaatgtcttttccggcaatactccggcaaccggccaccggagtttccaccggcgatcccgtcgccggagaaaacttgttctcgaacacaaaattcctcctgaacaatatctccggcgaggcacgggacggcgagatagtcgccgtcctgggtgcatcagggtcggggaaatcgaccctgatcgatgccctcgcgaataggatcgcgaaggagagtttaaaaggaacgataacgttgaacggagagccacttgattcgagattattgaaagtaatctcagcatatgtaatgcaagatgatcttttatatccaatgttgacagttgaagagacgttaatgtttgcagctgaattcagattgccacgtactttgtcaaaatcaaaaaagaaaatgagagttcaagctttgattgatcaattaggactacgaaatgctgcaaaaacaatcattggtgatgaggtaagttatatatagagtataatgatgactacaaactgatcatatttatttttttaacatgcatttaataaaaatttactattttgaacagggtcatcgtggagtgtctggtggtgaaagacgacgagtttcgattggaattgatattattcatgaccctatcatattgtttttagacgaaccaacttcaggtcttgattcgactagtgcatacatggtggtgaaagttcttcaacgaattgctcaaagtggaagtattgtgatcatgtcaattcatcagccaagttatcgaattctcgggttattggatcggatgctcttcttgtcccgtggtcaaacggtttatagcgggtcacctatgaacctcccacatttttttgctgattttggtcacccaattccagatagtgaaaatcgaacagagtttgctctggatctgattcgggaactagaagggtccccaggagggacaaaaagtttggttgagttcaacaaaacatgggaaaatactaaaaggagtaatgaaaatcctggaaccctaacacctactcatggattgtcattgaaagaagcaattagcgcgagtatttcaagaggaaagttggtttcagggacaacgagtgatattcatacaagtccagcatcaatggttccaacttacgcgaatccattttggattgaaatggttgtcttgtccaagaggtcatttacaaattcttggagggtgccagagttgtttggtatccgtctaggggcaatcgtggtaacggggttcatcctagctaccatgttttggcaacttgatgattcccctaaaggggttcaagaaaggcttggtttctttgcatttgctatgtcaacaactttctatacttgcgcggacgcgttgcctgtgttcctccaagagaggtacattttcatgagggagactgcttataatgcttataggagatcttcctattgtctatcgcatgctatagtttctttgccagcattgatctttcttagctttgcatttgccgctataactttttgggctgtaggccttgtaggtggattttcgggctttttgttctatttcgcaataatactagcctccttttgggccgggaattcatttgtcacgttcctctccggtgtagttcctagtgtcatgttaggttacaccattgtggtcgcgatcctagcctatttcctcctcttctcaggattcttcatcaatcgcgataggattccaccttattggatatggtttcactacctatctctggtgaaatatccttatgaagctgtgttacaaaatgaatttgatgatgcaactaagtgttttgtcaaagggattcaattgtttgataattcaccacttggaaatgtgcctaatgcattgaaggaaaaattgttgagtacaatgagtaacacattaaatgtcaaaattacaagttcaacatgtgtgactactggggctgatatattggttcaacaagggattactgatttaagtaagtggaattgtttgtggattactattgcatgggggtttttcttcagggttttgttttactttagcttgttgcttggaagtaagaacaagagaaggtga

SEQ ID NO: 8 - DNA, 2346 bp, Solanum tuberosum
atgtcaaggatagtagcggaaaatatgttacaagggggagaaaatgtacaattttataatcaaagagtacaacaagccatggagatgtcacaagccagcgcgtactcttcacccaccctaggccaaatgctaaagcgcgtgggagacgtgagaaaggaagccaccggcgacgaaactccggtgcaccggattctcgatatgagtgatactcaaagcatatcatctcactctcttccttttgtactctccttcaacaacctcacctacagcgtaaaagtccgccggaaaatgccttttccagcgatactccggcgaccggccgccggagtttccaccggtgatcccatcgccggagaaaatctgttcacgaacacaaaattcctcctgaacaatatctccggcgaggcccgggacggcgagatagtcgccgtcctgggtgcatcagggtcggggaaatcgaccctgatcgatgccctcgcgaataggatcgcgaaggagagtttaaaaggaacgataacgttaaacggagagccacttgattcgagattgttgaaagtaatctcagcatatgtaatgcaagatgatcttttatatccaatgttgacagttgaagaaacattaatgtttgcagctgaattcagattgccacgtacttcatcaaaatcaaaaaagaaaatgagagttcaagctttgattgatcaattaggactacgaaatgctgcaaaaacaatcattggtgatgaggtaacgttatatatacagtataatttttcatcgatgcctacaaactgatcattttttttttaacatttaataaaaatttactattttgaacagggtcatcgtggagtgtctggtggtgaaagacgacgagtttcgattggaattgatattattcatgaccctatcatattgtttttagacgagccaacttcaggtcttgactcgactagtgcatatatggtggtgaaggttctacaacgaattgctcaaagtggaagtattgttatcatgtcaattcatcagccaagttatcgaattctcgggttattggatcggatgctcttcttgtcccgtggtcaaacggtttatagtgggtcacctatgaacctcccacatttttttgctgattttggtcacccaataccggatagtgaaaatagaacagagtttgctctggatctaattcgcgaactagaagggtcccctggagggacaaaaagtttggttgagttcaacaaaacatgggaaaatactaaaaggagtaatgaaaatcctgaaatccaaacacctactcatggattgtcattgaaagaagcaattagcgcgagtatttcaagagggaagttggtttcagggacaacgagtgatattcatactagtccagcatcaatggttccaacttacgcgaatccattttggattgaaatgcttgtgttgtccaagagatcatttacgaattcttggagggtgccagagttatttggtattcgtctaggggcaatcgtggtcacggggttcatcctagctaccatgttttggcaacttgatgattcccctaaaggggttcaagaaaggcttggtttctttgcatttgctatgtcaacaactttctatacttgcgcggacgcgttgcctgtgtttctccaagagaggtacattttcatgagggagactgcttataatgcttataggagatcttcctattgtctatctcatgctatagtttctttgccagcattgatctttcttagctttgcatttgctgctataactttttgggctgtaggccttgtaggtggattttcgggctttttgttctatttcgcgataatactagcctccttctgggccgggaattcatttgtcacgttcctctccggtgtagttcctagtgtcatgttaggttacaccatagtggtcgcgatcctagcctatttcctcctcttctcaggattcttcatcaatcgcgataggattccaccttattggatatggtttcactacctgtctctggtgaaatatccttatgaagctgtgttacaaaatgaatttgatgatgcaacaaagtgttttgtcaaagggattcaattgtttgataattcaccacttggaaatgtgcctaatgcattgaaggaaaaattgttgagtacaatgagtaacacattaaatgtcaaaattacaagttcaacatgtgtgactactggggctgatatattggttcaacaagggattactgatttaagtaagtggaattgtttgtggattactattgcatgggggtttttctttagggttttgttttactttagcttgttgcttggaagtaagaacaagagaaggtga

SEQ ID NO: 9 - DNA, 2145 bp, Solanum lycopersicum
atgtcaaaagttgtagctcaaaatgtttcaccggtaagagattgtgtacctctttatgatcggagacagacggtagaaatgtcgtcgccgacgtttgctcagttgttgaacaacgtcggagatcacgtcgccggcgacgaaactgaaactccagttcaccatgttctgacgatgcagccgcagaactctattccgtttgtactgtcattcagcaacctcacatacagcgtgacagttcgccggaaaaacatttttccggcaatgtctaggggcttgacagaggaagcgccggtcacgaggacgaaagtgcttttacatgatatatcaggggaggcgcgtgatggagagctactcgcagtgctcggcgcgtctggttcggggaaatcgacgttgatcgatgctttagctaatcgaattgcgaaggagagcttgaaaggggagataaaattgaacggtgagaaattgcatacgaagttgctgaaagtgatatcggcgtacgtaatgcaagacgatctcctttatccgatgcttacagttgaagaaactttaatgttctcagcagagtttcgccttccacgaactctatcgaaatcgaagaagaaaagtagagttcaagcattaatcgatcaattaggactcagaaacgccgccaaaactataatcggcgatgaaggacaccgaggagtctccggcggtgaaagacggagagtttcgatcggaatcgacatcatccatgatccgatcatcttatttctcgatgaacctacttccggtctcgattccaccaatgctttcatggtggttaaagttcttcaacgaatcgcacagagcgggagtattgtgataatgtcgattcatcagccaagtaatcgaattctcggtttacttgatcgattaatcttcttatcacgtggacaaactgtttacagtgattcaccgttcaatttgccccaatttttcgctgattttggtcaatcaattccagaaaacgagaacagaacagagttcgcccttgatttaatccgcgaactcgaaggcacacctgatggaacaaataatttagttgaattcaaccgaaaatggaaaagttcttcaacaacattgacaatatatgatctatcactaaaggaagcaataagtgcaagcatttccagaggaaaattggtttctggtgctgctaatcctacctctatggttcctacttttgcaaatccaatatggaccgaaatggcagtactgtcaaagcgttcattcacaaactcatggcgtatgccggagattttcttcgtccgtttcagtgcagtaatggtcacgggttttatcctctcaaccattttctggcggctcgacaattcccctaaaggcatatgggaacgccttgggtttatagcattcgcagtgtcaacaacttactatatatgcgcggaggcgttgcccgttttcattcacgataggtacattttcatgagggaaacagcttacaatgcttatcggagatcatcatattgcctctctcacgctttggtttcgctaccatcattgataatcctctctctatcgttcgcagccctgactttctgggctgttggcctagacggtggagcttcgggctttttgttctacttctctgtcgttctagcttcaatctgggcggggaattcattcgtcactttcctctctagtgtcgttcctcatgtcatgatcggttacacaatcgtagttgcattactcggatacttcctcctcttcagtggattctacatgaatcgcgataggatcccatcttactggatatggtttcattacctctccctcgtgaaatacccatatgaagcagtgttactgaacgaattcaaagaccccacgaaatgcttcgttcgtggggttcaattgttcgataacagccaattgggggatttaccgaattcaataaaggagaaattattggacaatatgagcgaaacgttgaacgcaacgataacaagttccacttgtttaacgagtggcgcagatgtattgatgcaacaagggataactcaactgaacaaatggaattgtttgtgggtaacaattgcatggggatttttctataggattttgttttatttcactttgttattgggaagtaaaaataaaaggaggtaa

SEQ ID NO: 10 - DNA, 2157 bp, Solanum lycopersicum
atgagtagaattgttgctcaaaatgtatcaccggtaagagattgcgtacctctttatgatcggagacaaatggtagaaatgtcatcgccgacgtttgctcagttgttgaacaacgtcggagatgatgtcaccggcgacgaaactggagcttcagttcaccgagttctgacaatggaactgcctctgcagcaatcgattccatttgtattgtcattcagtaacctcacatacagcgtgagagtccgccggaaaaatatttttccggcgatgtccggccgccggaaccggacagatgaaccgcggtgcacgaggacgaaggtgcttttgaatgatatatcaggagaggcgcgcgacggagaactactcgcggtgctcggcgcgtctggttcggggaaatcgacgttgatcgatgctttagctaatcgaatttcgaaagatagcttgaaaggggagatgaaattgaacggtgagccattgcattcgaagttgctgaaagtgatatcggcgtacgtaatgcaagacgatctcctttatccgatgcttacagttgaagaaactttaatgttctcagcagagtttcgccttccacgaactctatcgaaatcgaagaagaagagtagagttcaagcattaatcgatcaattaggactccgaaacgccgccaaaactataatcggcgatgaaggccaccgaggagtctccggcggtgaaagacggagagtttcgatcggaatcgacatcatccatgatccgatcatcttattcctcgatgaacctacctccggtctcgattccaccagtgcttttatggtggttaaagttcttcaacgaatcgcacagagcggaagtattgtgataatgtcgattcatcaaccaagttatcgaattctcggtttacttgatcgattaatcttcttatcacgcggacagactgtttacagtggttcaccgttcaatttgccccaatttttcgctgattttggtcatccaattccagaaaacgagaataaaacagagttcgcgcttgatttaatccgcgaactcgaagggtcacctaatggaacaagtagtttagttgaattcaatcgaaaatggaaaaattcttcaacaacatcgacaatatatgatctatcactaaaggaagcaataagtgcaagcatttccagaggaaaattggtttctggtgctgctaatcctacgtctatggttcctacttttgcaaatccaatatggaccgaaatggcagtactatcaaaccgatcattcacaaactcatggcgtatgccggagattttcgctgtacgtttcggtgcagtaatggtcacgggtttcatcctcgccaccatgttctggcggctcgatgattcccctagaggtgtaagggaacggattgggtttttcgcgtttgcaatgtctacaacttactatacatgcgcggaggcattgcccgttttcattcacgagaggttcattttcatgagggaaacggcttacaatgcttatcggagatcatcatattgcctctctcacgctttggtttcgataccatcattggtattcctctctctatcgttcgcagctctgactttctgggctgttggcctagacggtggagcttcgagctttctcttctatttatctgtcgtccttgcttccttttgggctggtaactcattcgtcacgttcctctctggtgtcattcctcatgtcatgattggatacgtaatcgttgtagcaattttcgcgtattaccttctcttcagtggtttcttcttgaatcgtgataggattccgtcttactggatatggtttcattacatctcgctcgtgaaatacccatatgaagcagtgttgcagaacgaattcaaagatcccatgaaatgcttcgttcgtgggattcaattgttcgataacagcccactcggggatgttccgatttcgttgaaggagaaattgttggacagtataagcaatacgttgaacgtaaggataacaagttcgacatgtgtggtgactggtgcagatatattggtgcaacaagggataactcaactgaacaaatggaattgcttgtgggtaacaattgcatggggatttttctttaggattttgttttatttcactttgttattgggaagtaaaaataaaaggaggtaa

SEQ ID NO: 11 - DNA, 2193 bp, Nicotiana tabacum
atgtcaagaataattaatgcatcaccagttagagatagcataccactttatgatcggagacaagttgtagaaatgtcatcgccgacgtttggtcagttgttgaagaacgtcggcgatgtcaccggcgacgacgaaagtccacttcatcaagctcttaccatggacccgcatcactctaatattccctttgtactcgcattcaacaacctcacatacagtgtgaaagtccgccggaaggtcaattttccggcgatctcacgtagccggagcagccgcagccccgctgaggaaatcccgtccaccagaacaaaggtgcttttgaatgacatctgtggagaagcgcgtgatggagagctactcgcggttttaggggcgtctggttcgggaaaatcgacgctcattgatgcgttagcgaatcgtattgcgaaagatagcttgaaagggacagtaacactgaacggcgagccactgcactcgaaattactcaaagtcatatcggcttacgtaatgcaagacgatcttctctacccaatgcttacagtagaagaaacgttaatgttcgcagctgagtttaggcttccacggagtctatcaaaatcgaagaaaaaatccagagttcaagctttaatcgatcaattagggctcagaaacgccgcgaaaactatcatcggcgatgaaggtcaccgtggtgtctccggcggtgaacggcggcgcgtttcaatcggaattgacataatccatgacccgattattctctttctcgacgagccaacttcgggtctcgattccaccagtgcttttatggtgattaaagtacttcaacgaatcgcgcagagtggcagtattgtaattatgtcaatccatcagccgagttacagaattgtcggtttacttgaccggttgattttcctatcacgtggacaaactgtttacagtggctcgccgttgaatttgccacaattttttgctgattttggaaatccaatacctgaaaatgagaaccgcatagagttcgcgcttgatttaattcgcgaactcgaagggtcaggtaggacaaggagcttagtcgatttcaacaaaacatggcaacatatgaaacggactagtagtacaaatcagaatactgaaacaacaggtagaaatagaaatcgtttatcgttaaaggaagcgataagtgctagtatttctagaggaaaattggttcctggttcaacacatgttgctactagtcctacttctatggttcctacttttgcaaatccaatatggacagaaatagctgtactttcaaagcgatcattcactaactcgtggcgcatgcccgagatttttgctgttcgatttggtgcggttatggttacggggtttatactggctactatgttttggcgacttgacagttcacctaaaggtgtacaagaacggcttggattcttcgcgttcgcgatgtcaacgacttactatacatgtgcggacgcattgcccgtttttattcaagaaaggtacattttcatgagggaaacggcttataatggatatagaagatcatcttattgtctttctcatgctttgacttcgataccagcgttgattttcctcgctctgtcattcgccgccgtgacattctgggctgttggcttagatggtggattttctagctttttgttctatttcactgtcattttggcttcgttttgggcagggaattcattcgttacgttcctttctggtgtcgtgcctcatgtcatgctcggatacacaatcgtggtagcaattttagcctacttcctactctttagtggattcttcatgaatcgtgatagaatcccgtcttactggatctggttccattatatttcgctagtgaaatacccgtatgaagctgtgttacagaacgaatttgatgatcccacgaaatgcttcgttcgtgggattcaaatgttcgataacagtccacttggggctgttccgaattcgttaaaggagaagttgttgagcagtattagcagtacattgaatatgaggattacaagttcaacatgtgtgactactggatcagatatattggtgcaacaagggattacacaattgagcaagtggaattgcctttgggtaactattgcatgggggtttttgtttaggattttgttttatttctgcttgttgcttggaagtaagaataagagaagttaa

SEQ ID NO: 12 - protein, 707 aa, Brassica napus
MPRVSAESQEISFDGGSEPTLGELLKDFDGGDRKKNSGEDASTHHILDLTSPEIRPVPFLLSFNNLSYDIVHRRRFDFSRGKPASVKTLLNDVSGEACDGDILAVLGASGAGKSTLIDALAGRVSSLRGTVTLNGEKILQTRLLKVISAYVMQDDLLFPMLTVKETLMFASEFRLPRSLSKSKKMERVQTLIDQLGLRNAADTIIGDEGHRGVSGGERRRVSIGIDIIHDPILLFLDEPTSGLDSTNAFMVVQVLKRIARSGSIVIMTIHQPSARVLDLLDRLIILSRGKNVFSGSPTSLPQFLSDFGHPIPEKENITEFALDLVRQLEGSSEGTRELVKFNEKWQQNQSARATPMTTPYQALSLKESITASVSRGKLVSGSTSSNPISMDSVSSYANPPLVETFILAKRYMKNWIRTPELLGTRIATVMVTGLLLATIYWRLDNTPRGAQERMAFFAFGMSTMFYVCADNVPVFLQERFIFLRETTRNAYRTSSYVISHSLVSLPQLLALSIAFAATTFWTVGLSGGLESFLYYCLIIYAGFWSGSSFVTFVSGLVPNVMISFMITIAYLSYCLLLGGFYINRDRIPVYWIWFHYISLLKYPYEAVLINEFDDPSRCFVRGVQVFDGTLLAKVPDAMKVKLLDTLSSSLGTTITESTCLRTGPDLLMQQGISQLSKWDCLWITLAWGLFFRILFYFSLLFGSKNKR

SEQ ID NO: 13 - protein, 707 aa, Brassica napus
MPRVSAESQEISFDGGNEPTLGELLKDFDGGDRKKNSGEDASTHHILDLTSPETRPVPFLLSFNNLSYDIVHRRRFVFSRGKPASVKPLLNDVSGEACDGDILAVLGASGAGKSTLIDALAGRVGSLRGTVTLNGEKILQTRLLKVISAYVMQDDLLFPMLTVKETLMFASEFRLPRSLSKSKKMERVQTLIDKLGLRNAADTIIGDEGHRGVSGGERRRVSIGIDIIHDPILLFLDEPTSGLDSTNAFMVVQVLKRIARSGSIVIMTIHQPSARVLDLLDRLIILSRGENVFSGSPTSLPQFLSDFGHPIPEKENITEFALDLVRQLEGSSEGTRELVEFNEKWQQNHSARATPMTTPYQALSLKESITASVSRGKLVSGSTSSDPISMDSVSSYANPPLVETFILAKRYMKNWIRTPELIGTRIATVMVTGLLLATIYWRLDNTPRGAQERMAFFAFGMSTMFYVCADNVPVFLQERFIFLRETTRNAYRTSSYVISHSLVSLPQLLALSIAFAATTFWTVGLSGGLESFLYYCLIIYAGFWSGSSFVTFVSGLVPNVMISFMITIAYLSYCLLLGGFYINRDRIPVYWIWFHYISLLKYPYEAVLINEFDDPSRCFVRGVQVFDGTLLAKVPDAMKVKLLDTLSSSLGTTITESTCLRTGPDLLMQQGISQLSKWDCLWITLAWGLFFRILFYFSLLFGSKNKR

SEQ ID NO: 14 - protein, 750 aa, Brassica napus
MSRIVAENMLQGGENVQFYDQRVQQAMEMSQASAYSSPTLGQMLKRVGDVRKEVTGDETPVHRILDMSDTQSISSHSLPFVLSFNNLTYSVKVRRKMSFPAILRQPATGVSTGDPVAGENLFSNTKFLLNNISGEARDGEIVAVLGASGSGKSTLIDALANRIAKESLKGTITLNGEPLDSRLLKVISAYVMQDDLLYPMLTVEETLMFAAEFRLPRTLSKSKKKMRVQALIDQLGLRNAAKTIIGDEGHRGVSGGERRRVSIGIDIIHDPIILFLDEPTSGLDSTSAYMVVKVLQRIAQSGSIVIMSIHQPSYRILGLLDRMLFLSRGQTVYSGSPMNLPHFFADFGHPIPDSENRTEFALDLIRELEGSPGGTKSLVEFNKTWENTKRSNENPGTLTPTHGLSLKEAISASISRGKLVSGTTSDIHTSPASMVPTYANPFWIEMVVLSKRSFTNSWRVPELFGIRLGAIVVTGFILATMFWQLDDSPKGVQERLGFFAFAMSTTFYTCADALPVFLQERYIFMRETAYNAYRRSSYCLSHAIVSLPALIFLSFAFAAITFWAVGLVGGFSGFLFYFAIILASFWAGNSFVTFLSGVVPSVMLGYTIVVAILAYFLLFSGFFINRDRIPPYWIWFHYLSLVKYPYEAVLQNEFDDATKCFVKGIQLFDNSPLGNVPNALKEKLLSTMSNTLNVKITSSTCVTTGADILVQQGITDLSKWNCLWITIAWGFFFRVLFYFSLLLGSKNKRR

SEQ ID NO: 15 - protein, 749 aa, Solanum tuberosum
MSRIVAENMLQGGENVQFYNQRVQQAMEMSQASAYSSPTLGQMLKRVGDVRKEATGDETPVHRILDMSDTQSISSHSLPFVLSFNNLTYSVKVRRKMPFPAILRRPAAGVSTGDPIAGENLFTNTKFLLNNISGEARDGEIVAVLGASGSGKSTLIDALANRIAKESLKGTITLNGEPLDSRLLKVISAYVMQDDLLYPMLTVEETLMFAAEFRLPRTSSKSKKKMRVQALIDQLGLRNAAKTIIGDEGHRGVSGGERRRVSIGIDIIHDPIILFLDEPTSGLDSTSAYMVVKVLQRIAQSGSIVIMSIHQPSYRILGLLDRMLFLSRGQTVYSGSPMNLPHFFADFGHPIPDSENRTEFALDLIRELEGSPGGTKSLVEFNKTWENTKRSNENPEIQTPTHGLSLKEAISASISRGKLVSGTTSDIHTSPASMVPTYANPFWIEMLVLSKRSFTNSWRVPELFGIRLGAIVVTGFILATMFWQLDDSPKGVQERLGFFAFAMSTTFYTCADALPVFLQERYIFMRETAYNAYRRSSYCLSHAIVSLPALIFLSFAFAAITFWAVGLVGGFSGFLFYFAIILASFWAGNSFVTFLSGVVPSVMLGYTIVVAILAYFLLFSGFFINRDRIPPYWIWFHYLSLVKYPYEAVLQNEFDDATKCFVKGIQLFDNSPLGNVPNALKEKLLSTMSNTLNVKITSSTCVTTGADILVQQGITDLSKWNCLWITIAWGFFFRVLFYFSLLLGSKNKR

SEQ ID NO: 16 - protein, 713 aa, Solanum lycopersicum
MSKVVAQNVSPVRDCVPLYDRRQTVEMSSPTFAQLLNNVGDHVAGDETETPVHHVLTMQPQNSIPFVLSFSNLTYSVTVRRKNIFPAMSRGLTEEAPVTRTKVLLHDISGEARDGELLAVLGASGSGKSTLIDALANRIAKESLKGEIKLNGEKLHTKLLKVISAYVMQDDLLYPMLTVEETLMFSAEFRLPRTLSKSKKKSRVQALIDQLGLRNAAKTIIGDEGHRGVSGGERRRVSIGIDIIHDPIILFLDEPTSGLDSTNAFMVVKVLQRIAQSGSIVIMSIHQPSNRILGLLDRLIFLSRGQTVYSDSPFNLPQFFADFGQSIPENENRTEFALDLIRELEGTPDGTNNLVEFNRKWKSSSTTLTIYDLSLKEAISASISRGKLVSGAANPTSMVPTFANPIWTEMAVLSKRSFTNSWRMPEIFFVRFSAVMVTGFILSTIFWRLDNSPKGIWERLGFIAFAVSTTYYICAEALPVFIHDRYIFMRETAYNAYRRSSYCLSHALVSLPSLIILSLSFAALTFWAVGLDGGASGFLFYFSVVLASIWAGNSFVTFLSSVVPHVMIGYTIVVALLGYFLLFSGFYMNRDRIPSYWIWFHYLSLVKYPYEAVLLNEFKDPTKCFVRGVQLFDNSQLGDLPNSIKEKLLDNMSETLNATITSSTCLTSGADVLMQQGITQLNKWNCLWVTIAWGFFYRILFYFTLLLGSKNKR

SEQ ID NO: 17 - protein, 717 aa, Solanum lycopersicum
MSRIVAQNVSPVRDCVPLYDRRQMVEMSSPTFAQLLNNVGDDVTGDETGASVHRVLTMELPLQQSIPFVLSFSNLTYSVRVRRKNIFPAMSGRRNRTDEPRCTRTKVLLNDISGEARDGELLAVLGASGSGKSTLIDALANRISKDSLKGEMKLNGEPLHSKLLKVISAYVMQDDLLYPMLTVEETLMFSAEFRLPRTLSKSKKKSRVQALIDQLGLRNAAKTIIGDEGHRGVSGGERRRVSIGIDIIHDPIILFLDEPTSGLDSTSAFMVVKVLQRIAQSGSIVIMSIHQPSYRILGLLDRLIFLSRGQTVYSGSPFNLPQFFADFGHPIPENENKTEFALDLIRELEGSPNGTSSLVEFNRKWKNSSTTSTIYDLSLKEAISASISRGKLVSGAANPTSMVPTFANPIWTEMAVLSNRSFTNSWRMPEIFAVRFGAVMVTGFILATMFWRLDDSPRGVRERIGFFAFAMSTTYYTCAEALPVFIHERFIFMRETAYNAYRRSSYCLSHALVSIPSLVFLSLSFAALTFWAVGLDGGASSFLFYLSVVLASFWAGNSFVTFLSGVIPHVMIGYVIVVAIFAYYLLFSGFFLNRDRIPSYWIWFHYISLVKYPYEAVLQNEFKDPMKCFVRGIQLFDNSPLGDVPISLKEKLLDSISNTLNVRITSSTCVVTGADILVQQGITQLNKWNCLWVTIAWGFFFRILFYFTLLLGSKNKR

SEQ ID NO: 18 - protein, 730 aa, Nicotiana tabacum
MSRIINASPVRDSIPLYDRRQVVEMSSPTFGQLLKNVGDVTGDDESPLHQALTMDPHHSNIPFVLAFNNLTYSVKVRRKVNFPAISRSRSSRSPAEEIPSTRTKVLLNDICGEARDGELLAVLGASGSGKSTLIDALANRIAKDSLKGTVTLNGEPLHSKLLKVISAYVMQDDLLYPMLTVEETLMFAAEFRLPRSLSKSKKKSRVQALIDQLGLRNAAKTIIGDEGHRGVSGGERRRVSIGIDIIHDPIILFLDEPTSGLDSTSAFMVIKVLQRIAQSGSIVIMSIHQPSYRIVGLLDRLIFLSRGQTVYSGSPLNLPQFFADFGNPIPENENRIEFALDLIRELEGSGRTRSLVDFNKTWQHMKRTSSTNQNTETTGRNRNRLSLKEAISASISRGKLVPGSTHVATSPTSMVPTFANPIWTEIAVLSKRSFTNSWRMPEIFAVRFGAVMVTGFILATMFWRLDSSPKGVQERLGFFAFAMSTTYYTCADALPVFIQERYIFMRETAYNGYRRSSYCLSHALTSIPALIFLALSFAAVTFWAVGLDGGFSSFLFYFTVILASFWAGNSFVTFLSGVVPHVMLGYTIVVAILAYFLLFSGFFMNRDRIPSYWIWFHYISLVKYPYEAVLQNEFDDPTKCFVRGIQMFDNSPLGAVPNSLKEKLLSSISSTLNMRITSSTCVTTGSDILVQQGITQLSKWNCLWVTIAWGFLFRILFYFCLLLGSKNKRS

SEQ ID NO: 19 - DNA, 2172 bp, Artificial Sequence
  Description of Artificial Sequence Synthetic construct
atgaatctatcactcagcggtagaaagattgccatgacacgtgtttcggcggaaactcagtatatcactcccatcggatcaccaaccctcgacgagttgctgaaagactgcgacagtttccgaaaaggagattccggcgacggcgtaaaaagcgacgatcctgcacatcacataatagacgtcgaagccttgtacgtaaaacctgtcccgtacgtcttaaactttaacaatcttcaatacgatgtcacacttcgccggcggtttggcttctcacggcaaaacggagtaaagactctactcgatgatgtttccggagaggcttctgacggcgagctcctcgcggttttaggggcgtctggttcgggaaaatcgacgctcattgatgcgttagcgaatcgtattgcgaaagatagcttgaaagggacagtaacactgaacggcgagccactgcactcgaaattactcaaagtcatatcggcttacgtaatgcaagacgatcttctctacccaatgcttacagtagaagaaacgttaatgttcgcagctgagtttaggcttccacggagtctatcaaaatcgaagaaaaaatccagagttcaagctttaatcgatcaattagggctcagaaacgccgcgaaaactatcatcggcgatgaaggtcaccgtggtgtctccggcggtgaacggcggcgcgtttcaatcggaattgacataatccatgacccgattattctctttctcgacgagccaacttcgggtctcgattccaccagtgcttttatggtgattaaagtacttcaacgaatcgcgcagagtggcagtattgtaattatgtcaatccatcagccgagttacagaattgtcggtttacttgaccggttgattttcctatcacgtggacaaactgtttacagtggctcgccgttgaatttgccacaattttttgctgattttggaaatccaatacctgaaaatgagaaccgcatagagttcgcgcttgatttaattcgcgaactcgaagggtcaggtaggacaaggagcttagtcgatttcaacaaaacatggcaacatatgaaacggactagtagtacaaatcagaatactgaaacaacaggtagaaatagaaatcgtttatcgttaaaggaagcgataagtgctagtatttctagaggaaaattggttcctggttcaacacatgttgctactagtcctacttctatggttcctacttttgcaaatccaatatggacagaaatagctgtactttcaaagcgatcattcactaactcgtggcgcatgcccgagatttttgctgttcgatttggtgcggttatggttacggggtttatactggctactatgttttggcgacttgacagttcacctaaaggtgtacaagaacggcttggattcttcgcgttcgcgatgtcaacgacttactatacatgtgcggacgcattgcccgtttttattcaagaaaggtacattttcatgagggaaacggcttataatggatatagaagatcatcttattgtctttctcatgctttgacttcgataccagcgttgattttcctcgctctgtcattcgccgccgtgacattctgggctgttggcttagatggtggattttctagctttttgttctatttcactgtcattttggcttcgttttgggcagggaattcattcgttacgttcctttctggtgtcgtgcctcatgtcatgctcggatacacaatcgtggtagcaattttagcctacttcctactctttagtggattcttcatgaatcgtgatagaatcccgtcttactggatctggttccattatatttcgctagtgaaatacccgtatgaagctgtgttacagaacgaatttgatgatcccacgaaatgcttcgttcgtgggattcaaatgttcgataacagtccacttggggctgttccgaattcgttaaaggagaagttgttgagcagtattagcagtacattgaatatgaggattacaagttcaacatgtgtgactactggatcagatatattggtgcaacaagggattacacaattgagcaagtggaattgcctttgggtaactattgcatgggggtttttgtttaggattttgttttatttctgcttgttgcttggaagtaagaataagagaagttaa

SEQ ID NO: 20 - protein, 723 aa, Artificial Sequence
  Description of Artificial Sequence Synthetic construct
MNLSLSGRKIAMTRVSAETQYITPIGSPTLDELLKDCDSFRKGDSGDGVKSDDPAHHIIDVEALYVKPVPYVLNFNNLQYDVTLRRRFGFSRQNGVKTLLDDVSGEASDGELLAVLGASGSGKSTLIDALANRIAKDSLKGTVTLNGEPLHSKLLKVISAYVMQDDLLYPMLTVEETLMFAAEFRLPRSLSKSKKKSRVQALIDQLGLRNAAKTIIGDEGHRGVSGGERRRVSIGIDIIHDPIILFLDEPTSGLDSTSAFMVIKVLQRIAQSGSIVIMSIHQPSYRIVGLLDRLIFLSRGQTVYSGSPLNLPQFFADFGNPIPENENRIEFALDLIRELEGSGRTRSLVDFNKTWQHMKRTSSTNQNTETTGRNRNRLSLKEAISASISRGKLVPGSTHVATSPTSMVPTFANPIWTEIAVLSKRSFTNSWRMPEIFAVRFGAVMVTGFILATMFWRLDSSPKGVQERLGFFAFAMSTTYYTCADALPVFIQERYIFMRETAYNGYRRSSYCLSHALTSIPALIFLALSFAAVTFWAVGLDGGFSSFLFYFTVILASFWAGNSFVTFLSGVVPHVMLGYTIVVAILAYFLLFSGFFMNRDRIPSYWIWFHYISLVKYPYEAVLQNEFDDPTKCFVRGIQMFDNSPLGAVPNSLKEKLLSSISSTLNMRITSSTCVTTGSDILVQQGITQLSKWNCLWVTIAWGFLFRILFYFCLLLGSKNKRS

SEQ ID NO: 21 - protein, 699 aa, Oryza sativa
MATSAHTVLDVDSGGGAATAAAGPPVPYLLSFTDLSYSVRKGGGGVLSCLPSSRRRRHSNRLASADAPAPPDAPTKALLDGISGEARDGELFAVMGASGSGKSTLVDALAGRIARESLRGAVELNGEPLHGRRLRAISAYVMQDDLLYPMLTVRETLLFAAEFRLPRALSPDKKRARVDALIDQLGLARAADTIIGDEAHRGVSGGERRRVSIGTDIVHDPILLFLDEPTSGLDSASAFMVVQVLRRIAQSGSVVIMTIHQPSARILNILDRLLLLSRGRTVYAGTPVGLKPFFSEFGDPIPDNENPAEFALDTIRELEHQPDGAAPLADFNVKWQSMHAALPAADSKDSKRCTMPLELAITESVSRGKLVAGSGSGTASSTSVPTFANPLSVEVWVLMKRSFTNTGRMPELFVMRLGTIMVTGFILATIFWRLDDTPKGVQERLGFFAMAMSTMFYVCADALPVFVQERHIYLRETAHNAYRRLSYVFANAVVAFPPLVFLSLAFAVTTFFAVGLAGGGGSFLFFVLIILASFWAGSGFVTFLSAVVPHVMLGYTVVVAILAYFLLFSGFFINRDRIPSYWIWFHYLSLVKYPYQAVLQNEFRDATRCFSRGVEMFDGTPIGAMSRAVKLKVLDAISKTLGTNMTANTCVTTGADVLAQQAVTDIGKWKCLLVTVAWGFFFRALFYVVLLVGSKNKRR

SEQ ID NO: 22 - protein, 740 aa, Arabidopsis thaliana
MARIVAANDDDSMELNTISSIHDSTLGQLLKNVSDVRKMAIGDETPVHESLNQDYNDGYMRTVPFVLSFDNLTYNVSVRPKLDFRNLFPRRRTEDPEIAQTARPKTKTLLNNISGETRDGEIMAVLGASGSGKSTLIDALANRIAKGSLKGTVKLNGETLQSRMLKVISAYVMQDDLLFPMLTVEETLMFAAEFRLPRSLPKSKKKLRVQALIDQLGIRNAAKTIIGDEGHRGISGGERRRVSIGIDIIHDPILLFLDEPTSGLDSTSAFMVVKVLKRIAQSGSIVIMSIHQPSHRVLGLLDRLIFLSRGHTVYSGSPASLPRFFTEFGSPIPENENRTEFALDLIRELEGSAGGTRGLIEFNKKWQEMKKQSNRQPPLTPPSSPYPNLTLKEAIAASISRGKLVSGGESVAHGGATTNTTTLAVPAFANPMWIEIKTLSKRSMLNSRRQPELFGIRIASVVITGFILATVFWRLDNSPKGVQERLGFFAFAMSTMFYTCADALPVFLQERYIFMRETAYNAYRRSSYVLSHAIVSFPSLIFLSVAFAATTYWAVGLDGGLTGLLFYCLIILASFWSGSSFVTFLSGVVPSVMLGYTIVVAILAYFLLFSGFFINRNRIPDYWIWFHYMSLVKYPYEAVLQNEFSDATKCFVRGVQIFDNTPLGELPEVMKLKLLGTVSKSLGVTISSTTCLTTGSDILRQQGVVQLSKWNCLFITVAFGFFFRILFYFTLLLGSKNKRR

SEQ ID NO: 23 - DNA, 482 bp, Arabidopsis thaliana
  modified_base (36): a, c, g, t, unknown or other
  modified_base (66): a, c, g, t, unknown or other
atccaccactcgttttaacatcctgatacctcccgncggcgccaattaaaaattatatttatatanatattcactttattatatattttatttttataaataaattttatattttaaatatttattcttacattatattaaatttatttttttacataaaatataattattttttaaattattaaatattatatttattttttatttttatttatatttttataattatataattcatttttttttttttctattttatttataattttattttactcataattatttttatttattaatttttttttatatatttttttattttttattattattttatttttataattattatcattatatttttttattttaaattatttttatattttatttttattatatttatttctaaattttttatttttatttatttttttatttaaaatttattataattttatttcatttcttttattatt

SEQ ID NO: 24 - DNA, 359 bp, Solanum tuberosum
attacactcgagggacacgtatattttttattgtaatatagagaaaaagagtgaggtgtcgagttttgagaagggttacggtggtgatttgttatgggatgtcacgtgggaaagtttgagatgcagccggcgtgggccccgtgaagtagagcaatttgattggatgtttttgcaatttgaatgtttatttttggagttttatgggagtttgattgtaacctcttctttttctttgatattttctttcatgatcttttcttttgcctttgatattttcttgcaatagtactttactcttaactctggttaactgaattaatttgtctcaatttatttggttaacataagaattttggtac

SEQ ID NO: 25 - DNA, 1020 bp, Medicago sativa
tgcagggctttctctcaagatgattaattaattaattagtctcttctctttgtatgtgtttgaagggaatcaaagtgtagactcacaaagtgtgattttttatagaatggatgataaagatgtggattggtttttgtgtggctcagatgtgtggaagaatgtgtgatttagataatgtagatgtgccacagagtgcgtgggcttaaagtgggttatctcatcaggattgttgtgattggttggattggatgttatcctcctacgtggcttagattttttagtaaatataccgttttttttttttttttgttggagggattaatataccgttatttctcttatttgatctatgaaatataaaaatgagttaaatgactctatggggcaatatataggaaaagatcaaatgtcaaattaaatttgacaccaaattctaatcattaatacaactttaatccaactctctcatcaattcattaaataatcacatgtttaatcaaatgactcaacttttaattttaaaaataatttattctttctttttaatgacttgccaaaaattctctaatggtacatctctctaatgcgtcatccttatttatttacagcaattaacccttatttgtgtagcttttgtcaaattcctcaatgacatttgtacaaccattttggtacaacttttgtacaactttctctctcatactcacattatgtttttattctccctcttcctttttctctctctctctctattgtttttgaccaatgaaaagagagaacaacaaaattttctcaaatgttgtaacaaaattgttgtcaaaatatcactactcttattcttatcatccatatttctttactctaatgtttaataatgatttataacagtagtaggtgaatacttttgtccaaataaaaatgtgcacacttgctaacttaaacttttatgatttttgagttatttgacaacatataaatatatttttttaataatttctttctccacctacctaactggtc

SEQ ID NO: 26 - DNA, 494 bp, Nicotiana tabacum
ggatccttttgggagttatttatgctcccgtttggccattgattttggctactatttttcaagttaaattcttttttcaacttcccaaaaattgatttatgacattttttggataaaagtttttttccacctacaaaatttaacttctttttttcaaataaaatgcatgtccaaacacaacttcaactttcaaatatattttttaacataacttcaaaaactcttttttcaagttttaattatacatatgttcaactatgtattcatttctagttatgtttatcacgcatttcataagtgaatttcatacttatcttcatgcaaacatatatactataaaagatatattattcctaaatacaacatgtgatacgagatcattacattgcaactgaccttattatttttaaattttggacttcaccaaaaatagttgggttttttaatcgatttgatttaatttttcggtttggtgcggttttccgatttggttt

SEQ ID NO: 27 - DNA, 1024 bp, Solanum tuberosum
tcgagcacattgattgagttttatatgcaatatagtaataataataatatttcttataaagcaagaggtcaatttttttttattataccaacgtcactaaattatatttgataatgtaaaacaattcaattttacttaaatatcatgaaataaactatttttataaccaaattactaaatttttccaataaaaaaaagtcattaagaagacataaaataaatttgagtaaaaagagtgaagtcgactgacttttttttttttatcataagaaaataaattattaactttaacctaataaaacactaatataatttcatggaatctaatacttacctcttagaaataagaaaaagtgtttctaatagaccctcaatttacattaaatattttcaatcaaatttaaataacaaatatcaatatgaggtcaataacaatatcaaaataatatgaaaaaagagcaatacataatataagaaagaagatttaagtgcgattatcaaggtagtattatatcctaatttgctaatatttaaactcttatatttaaggtcatgttcatgataaacttgaaatgcgctatattagagcatatattaaaataaaaaaatacctaaaataaaattaagttatttttagtatatatttttttacatgacctacatttttctgggtttttctaaaggagcgtgtaagtgtcgacctcattctcctaattttccccaccacataaaaattaaaaaggaaaggtagcttttgcgtgttgttttggtacactacacctcattattacacgtgtcctcatataattggttaaccctatgaggcggtttcgtctagagtcggccatgccatctataaaatgaagctttctgcacctcatttttttcatcttctatctgatttctattataatttctctcaattgccttcaaatttctctttaaggttagaaatcttctctatttttggtttttgtctgtttagattctcgaattagctaatcaggtgctgttatagccctta

SEQ ID NO: 28 - DNA, 581 bp, Figwort mosaic virus
atttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaat

SEQ ID NO: 29 - protein, 18 aa, Artificial Sequence
  Description of Artificial Sequence Synthetic peptide
RIAKESLKGTITLNGEPL

SEQ ID NO: 30 - protein, 23 aa, Artificial Sequence
  Description of Artificial Sequence Synthetic peptide
VVPSVMLGYTIVVAILAYFLLFS

SEQ ID NO: 31 - protein, 725 aa, Arabidopsis thaliana
MNLSLSGRKIAMTRVSAETQYITPIGSPTLDELLKDCDSFRKGDSGDGVKSDDPAHHIIDVEALYVKPVPYVLNFNNLQYDVTLRRRFGFSRQNGVKTLLDDVSGEASDGDILAVLGASGAGKSTLIDALAGRVAEGSLRGSVTLNGEKVLQSRLLKVISAYVMQDDLLFPMLTVKETLMFASEFRLPRSLSKSKKMERVEALIDQLGLRNAANTVIGDEGHRGVSGGERRRVSIGIDIIHDPIVLFLDEPTSGLDSTNAFMVVQVLKRIAQSGSIVIMSIHQPSARIVELLDRLIILSRGKSVFNGSPASLPGFFSDFGRPIPEKENISEFALDLVRELEGSNEGTKALVDFNEKWQQNKISLIQSAPQTNKLDQDRSLSLKEAINASVSRGKLVSGSSRSNPTSMETVSSYANPSLFETFILAKRYMKNWIRMPELVGTRIATVMVTGCLLATVYWKLDHTPRGAQERLTLFAFVVPTMFYCCLDNVPVFIQERYIFLRETTHNAYRTSSYVISHSLVSLPQLLAPSLVFSAITFWTVGLSGGLEGFVFYCLLIYASFWSGSSVVTFISGVVPNIMLCYMVSITYLAYCLLLSGFYVNRDRIPFYWTWFHYISILKYPYEAVLINEFDDPSRCFVRGVQVFDSTLLGGVSDSGKVKLLETLSKSLRTKITESTCLRTGSDLLAQQGITQLSKWDCLWITFASGLFFRILFYFALLFGSRNKRT

(19) United States
(12) Patent Application Publication
(10) Pub. No.: US 2007/0074313 A1
(43) Pub. Date: Mar. 29, 2007

(54) Native antibiotic resistance genes

(75) Inventors: Caius Rommens, Boise, ID (US); Oleg V. Bougri, Boise, ID (US); Hua Yan, Boise, ID (US)

Correspondence Address: FOLEY AND LARDNER LLP;SUITE 500, 3000 K STREET NW, WASHINGTON, DC, 20007, US

(73) Assignee: J.R. SIMPLOT COMPANY

(21) Appl. No.: 11/521,588

(22) Filed: Sep. 15, 2006

Related U.S. Application Data
(60) Provisional application No. 60/717,245, filed on Sep. 16, 2005.

(51) Int. Cl. A01H 1/00; C07H 21/04; C12N 15/82; C12N 5/04; C07K 14/415

(52) U.S. Cl. 800/288; 536/23.6; 530/370; 435/468; 435/419

(57) ABSTRACT

The present invention provides polynucleotide and polypeptide sequences isolated from plants that confer tolerance against a selection agent. Methods for identifying and using such sequences are also provided.

### CROSS-REFERENCE TO RELATED PATENT APPLICATIONS

This is a Non-Provisional U.S. regular application, which claims priority to U.S. Provisional Application Ser. No. 60/717,245 filed on Sep. 16, 2005, which is incorporated herein by reference.

### FIELD OF THE INVENTION

The present invention relates to polynucleotide and polypeptide sequences derived from plant species that function as selectable markers for transformation.

### BACKGROUND

Genetically engineered traits provide valuable alternatives to those available through conventional breeding. Such traits are introduced into plants by applying transformation methods to plants, plant tissues, or plant cells. The most broadly-used transformation method is based on the ability of certain bacteria such as Agrobacterium to transfer part of a plasmid DNA to plant cell nuclei. Upon transfer, such DNA fragments may stably integrate into the plant cell genome. Importantly, the efficiency of even the most efficient methods is often below 1%. Selection systems are therefore often required to identify the rare transformed cells, and allow these cells to proliferate and regenerate into whole plants.

Until now, almost all selectable marker genes are from either bacterial or synthetic origin. For instance, the broadly used neomycin phosphotransferase (nptII) gene is from bacterial origin, and the epsps gene was produced synthetically in the Monsanto laboratories.

There is public concern about the permanent introduction of bacterial or synthetic DNA into the genome of a food crop (Lusk et al., reference; Rommens et al., 2004). Therefore, it would be beneficial to use selectable marker genes that are isolated from well-known food crops such as potato or tomato. The present invention describes such genes and their utility in plant transformation technology.

### SUMMARY OF THE INVENTION

The invention provides genes that were isolated from food crops, encode ABC transporters, and can be used as new selectable marker genes.

In one embodiment, the encoded protein contains the amino acid motifs xx, and provides tolerance to kanamycin.

In one embodiment, the selectable marker gene that provides tolerance against kanamycin encodes a protein that shares at least 80% identity with SEQ ID Nos: 14-18.

In another embodiment, the selectable marker gene encodes a protein that shares at least 80% identity with SEQ ID Nos2, 12, 15, 17, and 18, and provides tolerance against cadmium.

In another embodiment, the selectable marker gene encodes a protein that shares at least 80% identity with SEQ ID NOs: 2 and 15, and provides tolerance against deoxynivalenol.

One aspect of the present invention is an ABC transporter gene, wherein the ABC transporter gene (i) does not comprise the sequence of the Atwbc19 gene depicted in FIG. 1 or FIG. 2, but (ii) confers tolerance to a plant, when it is expressed in the plant, to a selection agent. In one embodiment, the encoded ABC transporter comprises the motif A[K/E][E/G]S and the selection agent is kanamycin. In another embodiment, the ABC transporter gene encodes a protein that shares at least 80% sequence identity with the protein encoded by a gene selected from the group consisting of SEQ ID NOs: 7-11. In another embodiment, the ABC transporter gene encodes a protein that shares at least 80% sequence identity with the protein encoded by a gene selected from the group consisting of SEQ ID NOs: 1, 8, 10, and 11, and wherein the selection agent is cadmium. In one embodiment, the ABC transporter gene encodes a protein that shares at least 80% sequence identity with the protein encoded by a gene selected from the group consisting of SEQ ID NOs: 1 and 8, and wherein the selection agent is deoxynivalenol.

In another aspect of the present invention is a method for designing a transformation selection system, comprising (i) producing a kill curve for a selection agent, (ii) identifying an ABC transporter that provides tolerance against the selection agent, and (iii) optimizing the selection system. In one embodiment, the selection agent is a toxin and selected from the group consisting of kanamycin, neomycin, paramomycin, geneticin, ampicillin, hygromycin, spectinomycin, streptomycin, glyphosate, chlorosulfuron, phosphinothricin, cadmium, zinc, copper, lead, aluminum, or iron. In another embodiment, the selection agent is a combination of at least two toxins.

In another aspect, a plant is provided comprising a gene that encodes a protein that shares at least 80% sequence identity with the protein encoded by a gene selected from the group consisting of SEQ ID NOs: 1 and 5-11, wherein the gene is operably linked to a foreign promoter and wherein at least one cell of that plant displays tolerance against at least one toxin.

### BRIEF DESCRIPTION OF THE DRAWINGS

FIG. 1. Alignment of Arabidopsis Atwbc19 and its Brassica napus homolog Krh1.

FIG. 2. Alignment of Atwbc19 and its potato homolog Krh4.

### DETAILED DESCRIPTION OF THE INVENTION

The present invention provides isolated polynucleotide and polypeptide sequences that were isolated from a food crop and can be used as selectable marker genes for transformation.

The present invention uses terms and phrases that are well known to those practicing the art. Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described herein are those well known and commonly employed in the art. Standard techniques are used for recombinant nucleic acid methods, polynucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. The techniques and procedures are generally performed according to conventional methodology (Molecular Cloning, A Laboratory Manual, 3rd. edition, edited by Sambrook & Russel Cold Spring Harbor Laboratory Press, Cold Spring Harbor, N.Y., 2001).

ABC Transporter: The ATP-binding cassette (ABC) transporters are transmembrane proteins that translocate a wide variety of substrates across extra- and intracellular membranes, including metabolic products, lipids and sterols, and drugs. In eukaryotes, ABC-transporters transport molecules to the outside of the plasma membrane or into membrane-bound organelles, e.g., the endoplasmic reticulum and mitochondria. ABC-transporters also exist within the placenta, implicating a protective role for the developing fetus against xenobiotics. Overexpression of ABC transporters can occur in cancer cell lines and tumors, which are multidrug resistant. Genetic variation in these ABC transporters genes is the cause or contributor to a wide variety of human disorders with Mendelian and complex inheritance including cystic fibrosis, neurological disease, retinal degeneration, cholesterol and bile transport defects, anemia, and drug response phenotypes. See Dean, The Human ATP-Binding Cassette (ABC) Transporter Superfamily, Bethesda (MD):National Library of Medicine, Nov. 18, 2002.

There are about 50 known ABC transporters present in humans, which are classified into seven families by the Human Genome Organization:

ABCA: 12 full transporters; responsible for transporting cholesterol and lipids; five of them are located in a cluster in the 17q24 chromosome.

ABCB: 4 full and 7 half transporters; some are located in the blood-brain barrier, liver, mitochondria and transports peptides and bile.

ABCC: 12 full transporters; ion transport, cell-surface receptors, toxin secretion. Includes the CFTR protein, which causes cystic fibrosis when deficient.

ABCD: 4 half transporters, which are all used in peroxisomes.

ABCE/ABCF: 1 ABCE and 3 ABCF proteins. These are ATP-binding domains which were derived from the ABC family but without the transmembrane domains. These proteins mainly regulate protein synthesis or expression.

ABCG: 6 “reverse” half-transporters, with the NBF at the NH3+ end and the TM at the COO− end. Transports lipids, bile, cholesterol, and other steroids.

Agrobacterium or bacterial transformation: as is well known in the field, Agrobacteria that are used for transforming plant cells are disarmed and virulent derivatives of, usually, Agrobacterium tumefaciens, Agrobacterium rhizogenes, that contain a vector. The vector typically contains a desired polynucleotide that is located between the borders of a T-DNA. However, any bacteria capable of transforming a plant cell may be used, such as, Rhizobium trifolii, Rhizobium leguminosarum, Phyllobacterium myrsinacearum, SinoRhizobium meliloti, and MesoRhizobium loti.

Angiosperm: vascular plants having seeds enclosed in an ovary. Angiosperms are seed plants that produce flowers that bear fruits. Angiosperms are divided into dicotyledonous and monocotyledonous plants.

Antibiotic Resistance: ability of a cell to survive in the presence of an antibiotic. Antibiotic resistance, as used herein, results from the expression of an antibiotic resistance gene in a host cell. A cell may have antibiotic resistance to any antibiotic.

Desired Polynucleotide: a desired polynucleotide of the present invention is a genetic element, such as a promoter, enhancer, or terminator, or gene or polynucleotide that is to be transcribed and/or translated in a transformed cell that comprises the desired polynucleotide in its genome. If the desired polynucleotide comprises a sequence encoding a protein product, the coding region may be operably linked to regulatory elements, such as to a promoter and a terminator, that bring about expression of an associated messenger RNA transcript and/or a protein product encoded by the desired polynucleotide. Thus, a “desired polynucleotide” may comprise a gene that is operably linked in the 5′-to 3′-orientation, a promoter, a gene that encodes a protein, and a terminator. Alternatively, the desired polynucleotide may comprise a gene or fragment thereof, in a “sense” or “antisense” orientation, the transcription of which produces nucleic acids that may affect expression of an endogenous gene in the plant cell. A desired polynucleotide may also yield upon transcription a double-stranded RNA product upon that initiates RNA interference of a gene to which the desired polynucleotide is associated. A desired polynucleotide of the present invention may be positioned within a T-DNA, such that the left and right T-DNA border sequences flank or are on either side of the desired polynucleotide. The present invention envisions the stable integration of one or more desired polynucleotides into the genome of at least one plant cell. A desired polynucleotide may be mutated or a variant of its wild-type sequence. It is understood that all or part of the desired polynucleotide can be integrated into the genome of a plant. It also is understood that the term “desired polynucleotide” encompasses one or more of such polynucleotides. Thus, a T-DNA of the present invention may comprise one, two, three, four, five, six, seven, eight, nine, ten, or more desired polynucleotides.

Dicotyledonous plant (dicot): a flowering plant whose embryos have two seed halves or cotyledons, branching leaf veins, and flower parts in multiples of four or five. Examples of dicots include but are not limited to, Eucalyptus, Populus, Liquidamber, Acacia, teak, mahogany, cotton, tobacco, Arabidopsis, tomato, potato sugar beet, broccoli, cassava, sweet potato, pepper, poinsettia, bean, alfalfa, soybean, carrot, strawberry, lettuce, oak, maple, walnut, rose, mint, squash, daisy, geranium, avocado, and cactus.

Endogenous: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Foreign: “foreign,” with respect to a nucleic acid, means that that nucleic acid is derived from non-plant organisms, or derived from a plant that is not the same species as the plant to be transformed or is not derived from a plant that is not interfertile with the plant to be transformed, does not belong to the species of the target plant. According to the present invention, foreign DNA or RNA represents nucleic acids that are naturally occurring in the genetic makeup of fungi, bacteria, viruses, mammals, fish or birds, but are not naturally occurring in the plant that is to be transformed. Thus, a foreign nucleic acid is one that encodes, for instance, a acide that is not naturally produced by the transformed plant. A foreign nucleic acide does not have to encode a protein product.

Gene: A gene is a segment of a DNA molecule that contains all the information required for synthesis of a product, polypeptide chain or RNA molecule that includes both coding and non-coding sequences.

Genetic element: a “genetic element” is any discreet nucleotide sequence such as, but not limited to, a promoter, gene, terminator, intron, enhancer, spacer, 5′-untraslated region, 3′-untranslated region, or recombinase recognition site.

Genetic modification: stable introduction of DNA into the genome of certain organisms by applying methods in molecular and cell biology.

Gymnosperm: as used herein, refers to a seed plant that bears seed without ovaries. Examples of gymnosperms include conifers, cycads, ginkgos, and ephedras.

Introduction: as used herein, refers to the insertion of a nucleic acid sequence into a cell, by methods including infection, transfection, transformation or transduction.

Kill curve: defines the frequency of shoot regeneration/explant for increasing concentrations of a chemical, whereby relatively high concentrations prevent regeneration, and result in eventual death of the explants. The lowest concentration of the chemical that prevents shoot regeneration is the minimal concentration that can be used to select for transformed plant cells, whereby the selectable marker gene is a gene that provides tolerance against the chemical, thus, allowing transgenic shoot formation. The optimized concentration of the chemical to be used for plant transformation experiments is a concentration that is higher than the minimal concentration but still allows the selectable marker gene to confer tolerance to the transformed cell to produce a transformed shoot and, consequently, a transformed plant.

Monocotyledonous plant (monocot): a flowering plant having embryos with one cotyledon or seed leaf, parallel leaf veins, and flower parts in multiples of three. Examples of monocots include, but are not limited to turfgrass, maize, rice, oat, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (kentucky bluegrass), Lolium spp. (ryegrass species including annual ryegrass and perennial ryegrass), Festuca arundinacea (tall fescue) Festuca rubra commutata (fine fescue), Cynodon dactylon (common bermudagrass varieties including Tifgreen, Tifway II, and Santa Ana, as well as hybrids thereof); Pennisetum clandestinum (kikuyugrass), Stenotaphrum secundatum (st. augustinegrass), Zoysia japonica (zoysiagrass), and Dichondra micrantha.

Native: nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species.

Native Antibiotic Resistance Gene: antibiotic resistance gene isolated from a plant species that is isolated either from the genome of a plant or plant species that is to be transformed or is isolated from a plant or species that is sexually compatible or interfertile with the plant species that is to be transformed, is “native” to, i.e., indigenous to, the plant species.

Native DNA: any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. For instance, a native DNA may comprise a point mutation since such point mutations occur naturally. It is also possible to link two different native DNAs by employing restriction sites because such sites are ubiquitous in plant genomes.

Native Nucleic Acid Construct: a polynucleotide comprising at least one native DNA.

Operably linked: combining two or more molecules in such a fashion that in combination they function properly in a plant cell. For instance, a promoter is operably linked to a structural gene when the promoter controls transcription of the structural gene.

P-DNA: a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterum in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. See Rommens et al. in WO2003/069980, US-2003-0221213, US-2004-0107455, and WO2005/004585, which are all incorporated herein by reference.

Phenotype: phenotype is a distinguishing feature or characteristic of a plant, which may be altered according to the present invention by integrating one or more “desired polynucleotides” and/or screenable/selectable markers into the genome of at least one plant cell of a transformed plant. The “desired polynucleotide(s)” and/or markers may confer a change in the phenotype of a transformed plant, by modifying any one of a number of genetic, molecular, biochemical, physiological, morphological, or agronomic characteristics or properties of the transformed plant cell or plant as a whole. Thus, expression of one or more, stably integrated desired polynucleotide(s) in a plant genome, may yield a phenotype selected from the group consisting of, but not limited to, increased drought tolerance, enhanced cold and frost tolerance, improved vigor, enhanced color, enhanced health and nutritional characteristics, improved storage, enhanced yield, enhanced salt tolerance, enhanced heavy metal tolerance, increased disease tolerance, increased insect tolerance, increased water-stress tolerance, enhanced sweetness, improved vigor, improved taste, improved texture, decreased phosphate content, increased germination, increased micronutrient uptake, improved starch composition, and improved flower longevity.

Plant tissue: a “plant” is any of various photosynthetic, eukaryotic, multicellular organisms of the kingdom Plantae characteristically producing embryos, containing chloroplasts, and having cellulose cell walls. A part of a plant, i.e., a “plant tissue” may be treated according to the methods of the present invention to produce a transgenic plant. Many suitable plant tissues can be transformed according to the present invention and include, but are not limited to, somatic embryos, pollen, leaves, stems, calli, stolons, microtubers, and shoots. Thus, the present invention envisions the transformation of angiosperm and gymnosperm plants such as turfgrass, wheat, maize, rice, barley, oat, sugar beet, potato, tomato, tobacco, alfalfa, lettuce, carrot, strawberry, cassava, sweet potato, geranium, soybean, oak, pine, fir, acacia, eucalyptus, walnut, and palm. According to the present invention “plant tissue” also encompasses plant cells. Plant cells include suspension cultures, callus, embryos, meristematic regions, callus tissue, leaves, roots, shoots, gametophytes, sporophytes, pollen, seeds and microspores. Plant tissues may be at various stages of maturity and may be grown in liquid or solid culture, or in soil or suitable media in pots, greenhouses or fields. A plant tissue also refers to any clone of such a plant, seed, progeny, propagule whether generated sexually or asexually, and descendents of any of these, such as cuttings or seed. Of particular interest are conifers such as pine, fir and spruce, monocots such as Kentucky bluegrass, creeping bentgrass, maize, and wheat, and dicots such as cotton, tomato, lettuce, Arabidopsis, tobacco, and geranium.

Plant transformation and cell culture: broadly refers to the process by which plant cells are genetically modified and transferred to an appropriate plant culture medium for maintenance, further growth, and/or further development. Such methods are well known to the skilled artisan.

Progeny: a “progeny” of the present invention, such as the progeny of a transgenic plant, is one that is born of, begotten by, or derived from a plant or the transgenic plant. Thus, a “progeny” plant, i.e., an “F1” generation plant is an offspring or a descendant of the transgenic plant produced by the inventive methods. A progeny of a transgenic plant may contain in at least one, some, or all of its cell genomes, the desired polynucleotide that was integrated into a cell of the parent transgenic plant by the methods described herein. Thus, the desired polynucleotide is “transmitted” or “inherited” by the progeny plant. The desired polynucleotide that is so inherited in the progeny plant may reside within a T-DNA construct, which also is inherited by the progeny plant from its parent. The term “progeny” as used herein, also may be considered to be the offspring or descendants of a group of plants.

Promoter: promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. As stated earlier, the RNA generated may code for a protein or polypeptide or may code for an RNA interfering, or antisense molecule.

A plant promoter is a promoter capable of initiating transcription in plant cells whether or not its origin is a plant cell. Exemplary plant promoters include, but are not limited to, those that are obtained from plants, plant viruses, and bacteria such as Agrobacterium or Rhizobium which comprise genes expressed in plant cells. Examples of promoters under developmental control include promoters that preferentially initiate transcription in certain tissues, such as xylem, leaves, roots, or seeds. Such promoters are referred to as tissue-preferred promoters. Promoters which initiate transcription only in certain tissues are referred to as tissue-specific promoters. A cell type-specific promoter primarily drives expression in certain cell types in one or more organs, for example, vascular cells in roots or leaves. An inducible or repressible promoter is a promoter which is under environmental control. Examples of environmental conditions that may effect transcription by inducible promoters include anaerobic conditions or the presence of light. Tissue specific, tissue preferred, cell type specific, and inducible promoters constitute the class of non-constitutive promoters. A constitutive promoter is a promoter which is active under most environmental conditions, and in most plant parts.

Polynucleotide is a nucleotide sequence, comprising a gene coding sequence or a fragment thereof, (comprising at least 15 consecutive nucleotides, preferably at least 30 consecutive nucleotides, and more preferably at least 50 consecutive nucleotides), a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, an RNA transcript (such as an mRNA) or a processed nucleotide sequence (such as a cDNA). The polynucleotide may comprise a sequence in either sense or antisense orientations.

An isolated polynucleotide is a polynucleotide sequence that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

Seed: a “seed” may be regarded as a ripened plant ovule containing an embryo, and a propagative part of a plant, as a tuber or spore. Seed may be incubated prior to Agrobacterium-mediated transformation, in the dark, for instance, to facilitate germination. Seed also may be sterilized prior to incubation, such as by brief treatment with bleach. The resultant seedling can then be exposed to a desired strain of Agrobacterium.

Selectable/screenable marker: a gene that, if expressed in plants or plant tissues, makes it possible to distinguish them from other plants or plant tissues that do not express that gene. Screening procedures may require assays for expression of proteins encoded by the screenable marker gene. Examples of selectable markers include the neomycin phosphotransferase (NPTII) gene encoding kanamycin and geneticin resistance, the hygromycin phosphotransferase (HPT or APHIV) gene encoding resistance to hygromycin, or other similar genes known in the art.

Sequence identity: as used herein, “sequence identity” or “identity” in the context of two nucleic acid or polypeptide sequences includes reference to the residues in the two sequences which are the same when aligned for maximum correspondence over a specified region. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, percentage of sequence identity means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE TO HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

Transcriptional terminators: The expression DNA constructs of the present invention typically have a transcriptional termination region at the opposite end from the transcription initiation regulatory region. The transcriptional termination region may be selected, for stability of the mRNA to enhance expression and/or for the addition of polyadenylation tails added to the gene transcription product. Translation of a nascent polypeptide undergoes termination when any of the three chain-termination codons enters the A site on the ribosome. Translation termination codons are UAA, UAG, and UGA.

In the instant invention, transcription terminators are derived from either a gene or, more preferably, from a sequence that does not represent a gene but intergenic DNA. Examples of such preferred and often more effective terminators include a T-rich sequence from Arabidopsis (SEQ ID NO: 23), a DNA fragment from potato (SEQ ID NO: 24), a DNA fragment from alfalfa (SEQ ID NO: 25) or a DNA fragment from tobacco (SEQ ID NO: 26).

Transfer DNA (T-DNA): an Agrobacterium T-DNA is a genetic element that is well-known as an element capable of integrating a nucleotide sequence contained within its borders into another genome. In this respect, a T-DNA is flanked, typically, by two “border” sequences. A desired polynucleotide of the present invention and a selectable marker may be positioned between the left border-like sequence and the right border-like sequence of a T-DNA. The desired polynucleotide and selectable marker contained within the T-DNA may be operably linked to a variety of different, plant-specific (i.e., native), or foreign nucleic acids, like promoter and terminator regulatory elements that facilitate its expression, i.e., transcription and/or translation of the DNA sequence encoded by the desired polynucleotide or selectable marker.

Transformation of plant cells: A process by which a nucleic acid is stably inserted into the genome of a plant cell. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols such as ‘refined transformation’ or ‘precise breeding’, viral infection, whiskers, electroporation, microinjection, polyethylene glycol-treatment, heat shock, lipofection and particle bombardment.

Transgenic plant: a transgenic plant of the present invention is one that comprises at least one cell genome in which an exogenous nucleic acid has been stably integrated. According to the present invention, a transgenic plant is a plant that comprises only one genetically modified cell and cell genome, or is a plant that comprises some genetically modified cells, or is a plant in which all of the cells are genetically modified. A transgenic plant of the present invention may be one that comprises expression of the desired polynucleotide, i.e., the exogenous nucleic acid, in only certain parts of the plant. Thus, a transgenic plant may contain only genetically modified cells in certain parts of its structure.

Variant: a “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, MD) software. “Variant” may also refer to a “shuffled gene” such as those described in Maxygen-assigned patents.

It is understood that the present invention is not limited to the particular methodology, protocols, vectors, and reagents, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a gene” is a reference to one or more genes and includes equivalents thereof known to those skilled in the art and so forth. Indeed, one skilled in the art can use the methods described herein to express any native gene (known presently or subsequently) in plant host systems.

Polynucleotide Sequences

The present invention relates to an isolated nucleic molecule comprising a polynucleotide having a sequence selected from the group consisting of any of the polynucleotide sequences of SEQ ID NOs: 1, 5, 7-11. The invention also provides protein sequences of SEQ ID NOs: 2, 12, 14-18. The invention further provides complementary nucleic acids, or fragments thereof, to any of the polynucleotide sequences of SEQ ID NOs: 1, 5, 7-11, as well as a nucleic acid, comprising at least 15 contiguous bases, which hybridizes to any of the polynucleotide sequences of SEQ ID NOs: 1, 5, 7-11.

By “isolated” nucleic acid molecule(s) is intended a nucleic acid molecule, DNA or RNA, which has been removed from its native environment. For example, recombinant DNA molecules contained in a DNA construct are considered isolated for the purposes of the present invention. Further examples of isolated DNA molecules include recombinant DNA molecules maintained in heterologous host cells or purified (partially or substantially) DNA molecules in solution. Isolated RNA molecules include in vitro RNA transcripts of the DNA molecules of the present invention. Isolated nucleic acid molecules, according to the present invention, further include such molecules produced synthetically.

Nucleic acid molecules of the present invention may be in the form of RNA, such as mRNA, or in the form of DNA, including, for instance, cDNA and genomic DNA obtained by cloning or produced synthetically. The DNA or RNA may be double-stranded or single-stranded. Single-stranded DNA may be the coding strand, also known as the sense strand, or it may be the non-coding strand, also referred to as the anti-sense strand.

Unless otherwise indicated, all nucleotide sequences determined by sequencing a DNA molecule herein were determined using an automated DNA sequencer (such as the Model 373 from Applied Biosystems, Inc.). Therefore, as is known in the art for any DNA sequence determined by this automated approach, any nucleotide sequence determined herein may contain some errors. Nucleotide sequences determined by automation are typically at least about 95% identical, more typically at least about 96% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence may be completely different from the amino acid sequence actually encoded by the sequenced DNA molecule, beginning at the point of such an insertion or deletion.

Each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U) where each thymidine deoxynucleotide (T) in the specified deoxynucleotide sequence in is replaced by the ribonucleotide uridine (U).

The present invention is also directed to fragments of the isolated nucleic acid molecules described herein. Preferably, DNA fragments comprise at least 15 nucleotides, and more preferably at least 20 nucleotides, still more preferably at least 30 nucleotides in length, which are useful as diagnostic probes and primers. Of course larger nucleic acid fragments of up to the entire length of the nucleic acid molecules of the present invention are also useful diagnostically as probes, according to conventional hybridization techniques, or as primers for amplification of a target sequence by the polymerase chain reaction (PCR), as described, for instance, in Molecular Cloning, A Laboratory Manual, 3rd. edition, edited by Sambrook & Russel., (2001), Cold Spring Harbor Laboratory Press, the entire disclosure of which is hereby incorporated herein by reference. By a fragment at least 20 nucleotides in length, for example, is intended fragments which include 20 or more contiguous bases from the nucleotide sequence of SEQ ID NOs: 1, 5, 7-11. The nucleic acids containing the nucleotide sequences listed in SEQ ID NOs: 1, 5, 7-11 can be generated using conventional methods of DNA synthesis which will be routine to the skilled artisan. For example, restriction endonuclease cleavage or shearing by sonication could easily be used to generate fragments of various sizes. Alternatively, the DNA fragments of the present invention could be generated synthetically according to known techniques.

In another aspect, the invention provides an isolated nucleic acid molecule comprising a polynucleotide which hybridizes under stringent hybridization conditions to a portion of the polynucleotide in a nucleic acid molecule of the invention described above. By a polynucleotide which hybridizes to a “portion” of a polynucleotide is intended a polynucleotide (either DNA or RNA) hybridizing to at least about 15 nucleotides, and more preferably at least about 20 nucleotides, and still more preferably at least about 30 nucleotides, and even more preferably more than 30 nucleotides of the reference polynucleotide. These fragments that hybridize to the reference fragments are useful as diagnostic probes and primers. A probe, as used herein is defined as at least about 100 contiguous bases of one of the nucleic acid sequences set forth in of SEQ ID NOs: 1, 5, 7-11. For the purpose of the invention, two sequences hybridize when they form a double-stranded complex in a hybridization solution of 6×SSC, 0.5% SDS, 5× Denhardt's solution and 100 μg of non-specific carrier DNA. See Ausubel et al., section 2.9, supplement 27 (1994). Sequences may hybridize at “moderate stringency,” which is defined as a temperature of 60° C. in a hybridization solution of 6×SSC, 0.5% SDS, 5× Denhardt's solution and 100 μg of non-specific carrier DNA. For “high stringency” hybridization, the temperature is increased to 68° C. Following the moderate stringency hybridization reaction, the nucleotides are washed in a solution of 2×SSC plus 0.05% SDS for five times at room ternperature, with subsequent washes with 0.1×SSC plus 0.1% SDS at 60° C. for 1 h. For high stringency, the wash temperature is increased to 68° C. For the purpose of the invention, hybridized nucleotides are those that are detected using 1 ng of a radiolabeled probe having a specific radioactivity of 10,000 cpm/ng, where the hybridized nucleotides are clearly visible following exposure to X-ray film at −70° C. for no more than 72 hours.

The present application is directed to such nucleic acid molecules which are at least 80%, 85%, 90%, 95%, 96%, 97%, 98%, 99% or 100% identical to a nucleic acid sequence described in of SEQ ID NOs: 1, 5, 7-11. Preferred, however, are nucleic acid molecules which are at least 95%, 96%, 97%, 98%, 99% or 100% identical to the nucleic acid sequence shown in of SEQ ID NOs: 1, 5, 7-11. Differences between two nucleic acid sequences may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to a reference nucleotide sequence refers to a comparison made between two molecules using standard algorithms well known in the art and can be determined conventionally using publicly available computer programs such as the BLASTN algorithm. See Altschul et al., Nucleic Acids Res. 25:3389-3402 (1997).

Sequence Analysis

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

The following running parameters are preferred for determination of alignments and similarities using BLASTN that contribute to the E values and percentage identity for polynucleotide sequences: Unix running command: blastall -p blastn -d embldb -e 10 -G0 -E0 -r 1 -v 30 -b 30 -i queryseq -o results; the parameters are: -p Program Name [String]; -d Database [String]; -e Expectation value (E) [Real]; -G Cost to open a gap (zero invokes default behavior) [Integer]; -E Cost to extend a gap (zero invokes default behavior) [Integer]; -r Reward for a nucleotide match (blastn only) [Integer]; -v Number of one-line descriptions (V) [Integer]; -b Number of alignments to show (B) [Integer]; -i Query File [File In]; and -o BLAST report Output File [File Out] Optional.

The “hits” to one or more database sequences by a queried sequence produced by BLASTN, FASTA, BLASTP or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, FASTA and BLASTP algorithms also produce “Expect” values for alignments. The Expect value (E) indicates the number of hits one can “expect” to see over a certain number of contiguous sequences by chance when searching a database of a certain size. The Expect value is used as a significance threshold for determining whether the hit to a database, such as the preferred EMBL database, indicates true similarity. For example, an E value of 0.1 assigned to a polynucleotide hit is interpreted as meaning that in a database of the size of the EMBL database, one might expect to see 0.1 matches over the aligned portion of the sequence with a similar score simply by chance. By this criterion, the aligned and matched portions of the polynucleotide sequences then have a probability of 90% of being the same. For sequences having an E value of 0.01 or less over aligned and matched portions, the probability of finding a match by chance in the EMBL database is 1% or less using the BLASTN or FASTA algorithm.

According to one embodiment, “variant” polynucleotides, with reference to each of the polynucleotides of the present invention, preferably comprise sequences having the same number or fewer nucleic acids than each of the polynucleotides of the present invention and producing an E value of 0.01 or less when compared to the polynucleotide of the present invention. That is, a variant polynucleotide is any sequence that has at least a 99% probability of being the same as the polynucleotide of the present invention, measured as having an E value of 0.01 or less using the BLASTN, FASTA, or BLASTP algorithms set at parameters described above.

Alternatively, variant polynucleotides of the present invention hybridize to the polynucleotide sequences recited in SEQ ID NOs: 1, 5, 7-11, or complements, reverse sequences, or reverse complements of those sequences, under stringent conditions.

The present invention also encompasses polynucleotides that differ from the disclosed sequences but that, as a consequence of the degeneracy of the genetic code, encode a polypeptide which is the same as that encoded by a polynucleotide of the present invention. Thus, polynucleotides comprising sequences that differ from the polynucleotide sequences recited in of SEQ ID NOs: 1, 5, 7-11; or complements, reverse sequences, or reverse complements thereof, as a result of conservative substitutions are contemplated by and encompassed within the present invention. Additionally, polynucleotides comprising sequences that differ from the polynucleotide sequences recited in of SEQ ID NOs: 1, 5, 7-11, or complements, reverse complements or reverse sequences thereof, as a result of deletions and/or insertions totaling less than 10% of the total sequence length are also contemplated by and encompassed within the present invention.

In addition to having a specified percentage identity to an inventive polynucleotide sequence, variant polynucleotides preferably have additional structure and/or functional features in common with the inventive polynucleotide. In addition to sharing a high degree of similarity in their primary structure to polynucleotides of the present invention, polynucleotides having a specified degree of identity to, or capable of hybridizing to an inventive polynucleotide preferably have at least one of the following features: (i) they contain an open reading frame or partial open reading frame encoding a polypeptide having substantially the same functional properties as the polypeptide encoded by the inventive polynucleotide; or (ii) they have domains in common.

Source of Elements and DNA Sequences

Any or all of the elements and DNA sequences that are described herein may be endogenous to one or more plant genomes. Accordingly, in one particular embodiment of the present invention, all of the elements and DNA sequences, which are selected for the ultimate transfer cassette are endogenous to, or native to, the genome of the plant that is to be transformed. For instance, all of the sequences may come from a potato genome. Alternatively, one or more of the elements or DNA sequences may be endogenous to a plant genome that is not the same as the species of the plant to be transformed, but which function in any event in the host plant cell. Such plants include potato, tomato, and alfalfa plants. The present invention also encompasses use of one or more genetic elements from a plant that is interfertile with the plant that is to be transformed.

In this regard, a “plant” of the present invention includes, but is not limited to angiosperms and gymnosperms such as potato, tomato, tobacco, avocado, alfalfa, lettuce, carrot, strawberry, sugarbeet, cassava, sweet potato, soybean, pea, bean, cucumber, grape, brassica, maize, turf grass, wheat, rice, barley, sorghum, oat, oak, eucalyptus, walnut, and palm. Thus, a plant may be a monocot or a dicot. “Plant” and “plant material,” also encompasses plant cells, seed, plant progeny, propagule whether generated sexually or asexually, and descendents of any of these, such as cuttings or seed. “Plant material” may refer to plant cells, cell suspension cultures, callus, embryos, meristematic regions, callus tissue, leaves, roots, shoots, gametophytes, sporophytes, pollen, seeds, germinating seedlings, and microspores. Plants may be at various stages of maturity and may be grown in liquid or solid culture, or in soil or suitable media in pots, greenhouses or fields. Expression of an introduced leader, trailer or gene sequences in plants may be transient or permanent.

In this respect, a plant-derived transfer-DNA (“P-DNA”) border sequence of the present invention is not identical in nucleotide sequence to any known bacterium-derived T-DNA border sequence, but it functions for essentially the same purpose. That is, the P-DNA can be used to transfer and integrate one polynucleotide into another. A P-DNA can be inserted into a tumor-inducing plasmid, such as a Ti-plasmid from Agrobacterum in place of a conventional T-DNA, and maintained in a bacterium strain, just like conventional transformation plasmids. The P-DNA can be manipulated so as to contain a desired polynucleotide, which is destined for integration into a plant genome via bacteria-mediated plant transformation. See Rommens et al. in WO2003/069980, US-2003-0221213, US-2004-0107455, and WO2005/004585, which are all incorporated herein by reference.

Thus, a P-DNA border sequence is different by 1, 2, 3, 4, 5, 6, 7, 8, 9, 10, 11, 12, 13, 14, 15, 16, 17, 18, 19, 20, or more nucleotides from a known T-DNA border sequence from an Agrobacterium species, such as Agrobacterium tumefaciens or Agrobacterium rhizogenes.

A P-DNA border sequence is not greater than 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51% or 50% similar in nucleotide sequence to an Agrobacterium T-DNA border sequence.

Methods were developed to identify and isolate transfer DNAs from plants, particularly potato and wheat, and made use of the border motif consensus described in US-2004-0107455, which is incorporated herein by reference.

In this respect, a plant-derived DNA of the present invention, such as any of the sequences, cleavage sites, regions, or elements disclosed herein is functional if it promotes the transfer and integration of a polynucleotide to which it is linked into another nucleic acid molecule, such as into a plant chromosome, at a transformation frequency of about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 15%, or about 5% or at least about 1%.

Any of such transformation-related sequences and elements can be modified or mutated to change transformation efficiency. Other polynucleotide sequences may be added to a transformation sequence of the present invention. For instance, it may be modified to possess 5′- and 3′-multiple cloning sites, or additional restriction sites. The sequence of a cleavage site as disclosed herein, for example, may be modified to increase the likelihood that backbone DNA from the accompanying vector is not integrated into a plant genome.

Any desired polynucleotide may be inserted between any cleavage or border sequences described herein. For example, a desired polynucleotide may be a wild-type or modified gene that is native to a plant species, or it may be a gene from a non-plant genome. For instance, when transforming a potato plant, an expression cassette can be made that comprises a potato-specific promoter that is operably linked to a desired potato gene or fragment thereof and a potato-specific terminator. The expression cassette may contain additional potato genetic elements such as a signal peptide sequence fused in frame to the 5′-end of the gene, and a potato transcriptional enhancer. The present invention is not limited to such an arrangement and a transformation cassette may be constructed such that the desired polynucleotide, while operably linked to a promoter, is not operably linked to a terminator sequence.

When a transformation-related sequence or element, such as those described herein, are identified and isolated from a plant, and if that sequence or element is subsequently used to transform a plant of the same species, that sequence or element can be described as “native” to the plant genome.

Thus, a “native” genetic element refers to a nucleic acid that naturally exists in, originates from, or belongs to the genome of a plant that is to be transformed. In the same vein, the term “endogenous” also can be used to identify a particular nucleic acid, e.g., DNA or RNA, or a protein as “native” to a plant. Endogenous means an element that originates within the organism. Thus, any nucleic acid, gene, polynucleotide, DNA, RNA, mRNA, or cDNA molecule that is isolated either from the genome of a plant or plant species that is to be transformed or is isolated from a plant or species that is sexually compatible or interfertile with the plant species that is to be transformed, is “native” to, i.e., indigenous to, the plant species. In other words, a native genetic element represents all genetic material that is accessible to plant breeders for the improvement of plants through classical plant breeding. Any variants of a native nucleic acid also are considered “native” in accordance with the present invention. In this respect, a “native” nucleic acid may also be isolated from a plant or sexually compatible species thereof and modified or mutated so that the resultant variant is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar in nucleotide sequence to the unmodified, native nucleic acid isolated from a plant. A native nucleic acid variant may also be less than about 60%, less than about 55%, or less than about 50% similar in nucleotide sequence.

A “native” nucleic acid isolated from a plant may also encode a variant of the naturally occurring protein product transcribed and translated from that nucleic acid. Thus, a native nucleic acid may encode a protein that is greater than or equal to 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, or 60% similar i amino acid sequence to the unmodified, native protein expressed in the plant from which the nucleic acid was isolated.

Promoters

The polynucleotides of the present invention can be used for specifically directing the expression of polypeptides or proteins in the tissues of plants. The nucleic acids of the present invention can also be used for specifically directing the expression of antisense RNA, or RNA involved in RNA interference (RNAi) such as small interfering RNA (siRNA), in the tissues of plants, which can be useful for inhibiting or completely blocking the expression of targeted genes. As used herein, “coding product” is intended to mean the ultimate product of the nucleic acid that is operably linked to the promoters. For example, a protein or polypeptide is a coding product, as well as antisense RNA or siRNA which is the ultimate product of the nucleic acid coding for the antisense RNA. The coding product may also be non-translated mRNA. The terms polypeptide and protein are used interchangeably herein. As used herein, promoter is intended to mean a nucleic acid, preferably DNA that binds RNA polymerase and/or other transcription regulatory elements. As with any promoter, the promoters of the current invention will facilitate or control the transcription of DNA or RNA to generate an mRNA molecule from a nucleic acid molecule that is operably linked to the promoter. The RNA may code for a protein or polypeptide or may code for an RNA interfering, or antisense molecule. As used herein, “operably linked” is meant to refer to the chemical fusion, ligation, or synthesis of DNA such that a promoter-nucleic acid sequence combination is formed in a proper orientation for the nucleic acid sequence to be transcribed into an RNA segment. The promoters of the current invention may also contain some or all of the 5′ untranslated region (5′ UTR) of the resulting mRNA transcript. On the other hand, the promoters of the current invention do not necessarily need to possess any of the 5′ UTR.

A promoter, as used herein, may also include regulatory elements. Conversely, a regulatory element may also be separate from a promoter. Regulatory elements confer a number of important characteristics upon a promoter region. Some elements bind transcription factors that enhance the rate of transcription of the operably linked nucleic acid. Other elements bind repressors that inhibit transcription activity. The effect of transcription factors on promoter activity may determine whether the promoter activity is high or low, i.e. whether the promoter is “strong” or “weak.”

In another embodiment, a constitutive promoter may be used for expressing the inventive polynucleotide sequences.

In another embodiment, a variety of inducible plant gene promoters can be used for expressing the inventive polynucleotide sequences. Inducible promoters regulate gene expression in response to environmental, hormonal, or chemical signals. Examples of hormone inducible promoters include auxin-inducible promoters (Baumann et al. Plant Cell 11:323-334(1999)), cytokinin-inducible promoter (Guevara-Garcia Plant Mol. Biol. 38:743-753(1998)), and gibberellin-responsive promoters (Shi et al. Plant Mol. Biol. 38:1053-1060(1998)). Additionally, promoters responsive to heat, light, wounding, pathogen resistance, and chemicals such as methyl jasmonate or salicylic acid, may be used for expressing the inventive polynucleotide sequences.

Nucleic Acid Constructs

The present invention provides constructs comprising the isolated nucleic acid molecules and polypeptide sequences of the present invention. In one embodiment, the DNA constructs of the present invention are Ti-plasmids derived from A. tumefaciens.

In developing the nucleic acid constructs of this invention, the various components of the construct or fragments thereof will normally be inserted into a convenient cloning vector, e.g., a plasmid that is capable of replication in a bacterial host, e.g., E. coli. Numerous vectors exist that have been described in the literature, many of which are commercially available. After each cloning, the cloning vector with the desired insert may be isolated and subjected to further manipulation, such as restriction digestion, insertion of new fragments or nucleotides, ligation, deletion, mutation, resection, etc. to tailor the components of the desired sequence. Once the construct has been completed, it may then be transferred to an appropriate vector for further manipulation in accordance with the manner of transformation of the host cell.

A recombinant DNA molecule of the invention typically includes a selectable marker so that transformed cells can be easily identified and selected from non-transformed cells. Examples of such markers include, but are not limited to, a neomycin phosphotransferase (nptll) gene (Potrykus et al., Mol. Gen. Genet. 199:183-188 (1985)), which confers kanamycin resistance. Cells expressing the nptli gene can be selected using an appropriate antibiotic such as kanamycin or G418. Other commonly used selectable markers include the bar gene, which confers bialaphos resistance; a mutant EPSP synthase gene (Hinchee et al., Bio/Technology 6:915-922 (1988)), which confers glyphosate resistance; and a mutant acetolactate synthase gene (ALS), which confers imidazolinone or sulphonylurea resistance (European Patent Application 154,204, 1985).

Additionally, vectors may include an origin of replication (replicons) for a particular host cell. Various prokaryotic replicons are known to those skilled in the art, and function to direct autonomous replication and maintenance of a recombinant molecule in a prokaryotic host cell.

The vectors will preferably contain selectable markers for selection in plant cells. Numerous selectable markers for use in selecting transfected plant cells including, but not limited to, kanamycin, glyphosate resistance genes, and tetracycline or ampicillin resistance for culturing in E. coli, A. tumefaciens and other bacteria.

For secretion of the translated protein into the lumen of the endoplasmic reticulum, the periplasmic space or into the extracellular environment, appropriate secretion signals may be incorporated into the expressed polypeptide. The signals may be endogenous to the polypeptide or they may be heterologous signals.

In one embodiment, a DNA construct of the current invention is designed in a manner such that a polynucleotide sequence described herein is operably linked to a tissue-specific promoter.

In a further embodiment, the DNA constructs of the current invention are desiged such that the polynucleotide sequences of the current invention are operably linked to DNA or RNA that encodes antisense RNA or interfering RNA, which corresponds to genes that code for polypeptides of interest, resulting in a decreased expression of targeted gene products. The use of RNAi inhibition of gene expression is described in U.S. Pat. No. 6,506,559, and the use of RNAi to inhibit gene expression in plants is specifically described in WO 99/61631, both of which are herein incorporated by reference.

The use of antisense technology to reduce or inhibit the expression of specific plant genes has been described, for example in European Patent Publication No. 271988. Reduction of gene expression led to a change in the phenotype of the plant, either at the level of gross visible phenotypic difference, for example a lack of lycopene synthesis in the fruit of tomato leading to the production of yellow rather than red fruit, or at a more subtle biochemical level, for example, a change in the amount of polygalacturonase and reduction in depolymerisation of pectins during tomato fruit ripening (Smith et. al., Nature, 334:724-726 (1988); Smith et. al., Plant Mol. Biol., 14:369-379 (1990)). Thus, antisense RNA has been demonstrated to be useful in achieving reduction of gene expression in plants.

In one embodiment an inventive polynucleotide sequence is capable of being transcribed inside a plant to yield an antisense RNA transcript is introduced into the plant, eg., into a plant cell. The inventive polynucleotide can be prepared, for example, by reversing the orientation of a gene sequence with respect to its promoter. Transcription of the exogenous DNA in the plant cell generates an intracellular RNA transcipt that is “antisense” with respect to that gene.

The invention also provides host cells which comprise the DNA constructs of the current invention. As used herein, a host cell refers to the cell in which the coding product is ultimately expressed. Accordingly, a host cell can be an individual cell, a cell culture or cells as part of an organism. The host cell can also be a portion of an embryo, endosperm, sperm or egg cell, or a fertilized egg.

Accordingly, the present invention also provides plants or plant cells, comprising the DNA constructs of the current invention. Preferably the plants are angiosperms or gymnosperms. The expression construct of the present invention may be used to transform a variety of plants, both monocotyledonous (e.g. wheat, turf grass, maize, rice, oat, wheat, barley, sorghum, orchid, iris, lily, onion, banana, sugarcane, and palm), dicotyledonous (e.g., Arabidopsis, potato, tobacco, tomato, avocado, pepper, sugarbeet, broccoli, cassava, sweet potato, cotton, poinsettia, legumes, alfalfa, soybean, pea, bean, cucumber, grape, brassica, carrot, strawberry, lettuce, oak, maple, walnut, rose, mint, squash, daisy, and cactus, oaks, eucalyptus, maple), and Gymnosperms (e.g., Scots pine; see Aronen, Finnish Forest Res. Papers, Vol. 595, 1996), white spruce (Ellis et al., Biotechnology 11:84-89, 1993), and larch (Huang et al., In Vitro Cell 27:201-207, 1991).

Plant Transformation and Regeneration

The present polynucleotides and polypeptides may be introduced into a host plant cell by standard procedures known in the art for introducing recombinant sequences into a target host cell. Such procedures include, but are not limited to, transfection, infection, transformation, natural uptake, electroporation, biolistics and Agrobacterium. Methods for introducing foreign genes into plants are known in the art and can be used to insert a construct of the invention into a plant host, including, biological and physical plant transformation protocols. See, for example, Miki et al., 1993, “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch et al., Science 227:1229-31, 1985), electroporation, micro-injection, and biolistic bombardment.

Accordingly, the present invention also provides plants or plant cells, comprising the polynucleotides or polypeptides of the current invention. In one embodiment, the plants are angiosperms or gymnosperms. Beyond the ordinary meaning of plant, the term “plants” is also intended to mean the fruit, seeds, flower, strobilus etc. of the plant. The plant of the current invention may be a direct transfectant, meaning that the vector was introduced directly into the plant, such as through Agrobacterium, or the plant may be the progeny of a transfected plant. The progeny may also be obtained by asexual reproduction of a transfected plant. The second or subsequent generation plant may or may not be produced by sexual reproduction, i.e., fertilization. Furthermore, the plant can be a gametophyte (haploid stage) or a sporophyte (diploid stage).

In this regard, the present invention contemplates transforming a plant with one or more transformation elements that genetically originate from a plant. The present invention encompasses an “all-native” approach to transformation, whereby only transformation elements that are native to plants are ultimately integrated into a desired plant via transformation. In this respect, the present invention encompasses transforming a particular plant species with only genetic transformation elements that are native to that plant species. The native approach may also mean that a particular transformation element is isolated from the same plant that is to be transformed, the same plant species, or from a plant that is sexually interfertile with the plant to be transformed.

On the other hand, the plant that is to be transformed, may be transformed with a transformation cassette that contains one or more genetic elements and sequences that originate from a plant of a different species. It may be desirable to use, for instance, a cleavage site, that is native to a potato genome in a transformation cassette or plasmid for transforming a tomato or pepper plant.

The present invention is not limited, however, to native or all-native approach. A transformation cassette or plasmid of the present invention can also comprise sequences and elements from other organisms, such as from a bacterial species.

### EXAMPLES

### Example 1

### Some of the Closest Homologs of Atwbc19 do not Confer Kanamycin Tolerance to Transgenic Plants

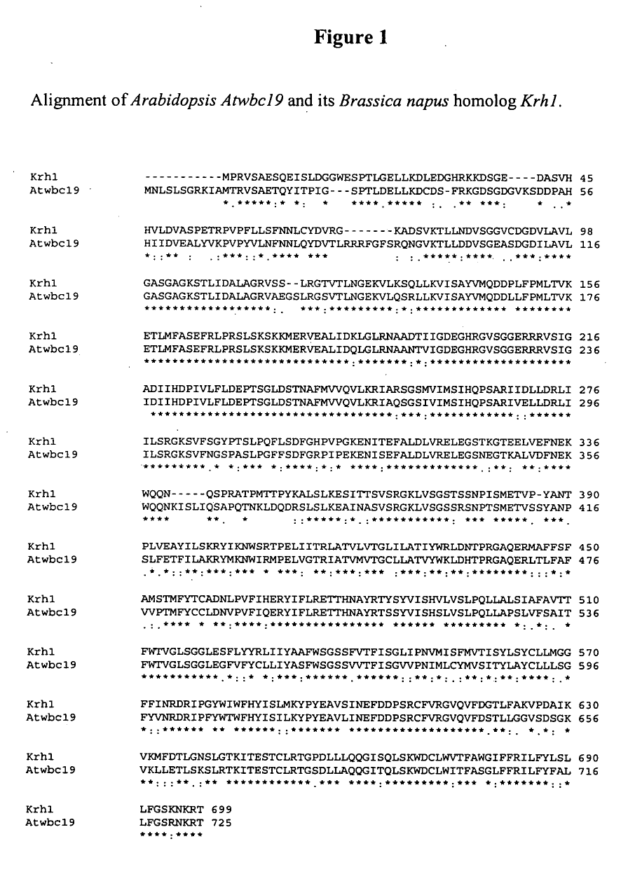

Overexpression of the Arabidopsis Atwbc19 gene was shown to result in kanamycin resistance in tobacco (Mentewab and Stewart Jr. Nat Biotechnol 23: 1177-1180, 2005). We therefore hypothesized that close homologs of this gene would also trigger resistance against this antibiotic if overexpressed in plants. To test this theory, we isolated the Atwbc19 homolog from a Brassica napa (rapeseed), a plant species that belongs to the same family (Cruciferae) as Arabidopsis. The kanamycin resistance gene homolog 1 (Krh1) is shown in SEQ ID NO.: 1, and its encoded protein (SEQ ID NO.: 2) displays 73% identity with Atwbc19 (FIG. 1).

The Krh1 gene was positioned between the 35S promoter of cauliflower mosaic virus (SEQ ID NO.: 3) and the terminator of the potato ubiquitin-3 gene (SEQ ID NO.: 4), and the resulting expression cassette was inserted between the two T-DNA borders of a pCAMBIA-derived binary vector (Genbank accession AF234297) to produce pSIM1073.

The binary vector pSIM106OD, which carries an expression cassette for the neomycin phosphotransferase (nptII) gene between T-DNA borders was used as control.

Both pSIM1073 and pSIM106OD were introduced into Agrobacterium tumefaciens LBA4404 or C58 cells as follows. Competent LB4404 cells (50 μL) were incubated for 5 min on ice in the presence of 1 μg of vector DNA, frozen for about 15 s in liquid nitrogen, and incubated at 37° C. for 5 min. After adding 1 mL of liquid broth, the treated cells were grown for 3 h at 28° C. and plated on liquid broth/agar containing streptomycin (100 mg/L) and kanamycin (100 mg/L). The vector DNAs were then isolated from overnight cultures of individual LBA4404 colonies and examined by restriction analysis to confirm the presence of intact plasmid DNA.

Instead of Agrobacterium tumefaciens, it is also possible to employ any bacterium that can be used to transform plants including, but not limited to, Rhizobium trifolii, Rhizobium leguminosarum, Phyllobacterium myrsinacearum, SinoRhizobium meliloti, and MesoRhizobium loti.

A 10-fold dilution of an ovemight-grown Agrobacterium culture was grown for 4 to 5 h, precipitated for 15 min at 3,800 rpm, washed with MS liquid medium (PhytoTechnology, Shawnee Mission, Kans.) supplemented with sucrose (3%, pH 5.7) and resuspended in the same medium to and optical density at 600 nm of 0.2 (for evaluation of new borders using pSIM-T vectors) or 0.04 (to assess the efficacy of new border-flanking DNA sequences). The suspension was then used to infect leaf explants of 3-week-old in vitro grown tobacco (Nicotiana tabacum) plants. Infected tobacco explants were incubated for 2 days on co-culture medium (one-tenth MS salts, 3% Suc, pH 5.7) containing 6 g/L agar at 25° C. in a Percival growth chamber (16-h light photoperiod) and subsequently transferred to M401/agar (PhytoTechnology) medium containing timentin (150 mg/L) and kanamycin (100 mg/L).

Two weeks later, explants that had been infected with the pSIM106OD strain contained many kanamycin resistant calli. However, employment of pSIM1073 did not yield any calli. Thus, overexpression of the Krh1 did not result in kanamycin resistance.

Two additional Atwbc19 homologs from Brassica napus, Krh2 (SEQ ID NO.: 5) and Krh3 (SEQ ID NO.: 6) were also tested for efficacy by inserting expression cassettes for these genes between T-DNA borders. The resulting binary vectors pSIM1074 and 1075 proved to lack any functional activity. This result demonstrates that the proteins encoded by Krh2 and Krh3, shown in SEQ ID NO.: 12 and 13, do not transport kanamycin. Collectively, our data demonstrate that Atwbc19 homologs do not necessarily display kanamycin resistance.

### Example 2

### A Distant Homolog of Atwbc19 from Potato Provides Kanamycin Tolerance

In addition to the Brassica Atwbc19 homologs, we had also isolated a more distant homolog from potato (Solanum tuberosum). This gene Krh4 (SEQ ID NO.: 7) encodes a protein that shares only 59% identity with Atwbc19 (SEQ ID NO.: 14). Although none of the Brassica genes displayed functional activity, we still tested the efficacy of a binary vector (pSIM1070) containing the potato gene. Interestingly, 42% of explants that were infected with an Agrobacterium strain carrying pSIM1070 developed kanamycin resistant calli. About 14% of these calli rooted on 100 mg L⁻¹ kanamycin.

Sequence alignments identified several exceptional amino acid regions that are only conserved between Atwbc19 and Krh4. Most importantly, these two kanamycin resistance proteins contain the sequence motif A[K/E][E/G]S at position 135-138 in Atwbc19 and 164-167 in Krh4. The proteins also contain F344, L495, F567, V598, Y606, S620, and T715 in Krh4, and the corresponding F315, L471, F551, V573, Y581, S595, and T690 in Atwbc19.

### Example 3

### Identification of Additional Kanamycin Resistance Genes from Solanaceous Crops

Based on these similarities, we isolated Krh5 from potato (SEQ ID NO.: 8), Krh6 from tomato (SEQ ID NO.: 9), Krh7 from tomato (SEQ ID NO.: 10), and Krh8 from tobacco (SEQ ID NO.: 11). Their predicted protein sequences are shown in SEQ ID NO.: 15-18, respectively. These genes were introduced into binary vectors to create pSIM1071, 1155, 1154, and 1152. A function tobacco transformation test demonstrated all four genes to confer kanamycin resistance to plants. Because the N-terminal region of Atwbc19 is most different from that of the Krh proteins, we produced a chimeric gene (SEQ ID NO.: 19) that encodes a protein with the N-terminus of Atwbc19 (328 base pairs) and the C-terminus of Krh8 (1842 base pairs) (SEQ ID NO.: 20). This chimeric gene proved equally effective as Krh8 itself, indicating that the specificity for kanamycin is not encoded by the N-terminal part of Atwbc19. A summary of transformation results is shown in FIG. 3.

Additional kanamycin resistance genes can be isolated from plant DNA by following the following procedures. First, databases can be searched for short regions that comprise amino acids conserved among Atwbc19 and Krh4-8. For instance, a BLAST search with the sequence ‘RIAKESLKGTITLNGEPL’ identifies the rice gene BAF1640 (SEQ ID NO.: 21). The alternative sequence ‘VVPSVMLGYTIVVAILAYFLLFS’ can be used to identify, for instance the Arabidopsis gene NP_{—}181467 (SEQ ID NO.: 22).

Second, the full length genes or cDNAs can be operably linked to a promoter and terminator, and the resulting expression cassettes can be positioned between T-DNA borders. Agrobacterium strains carrying binary vectors that contain these T-DNAs can then be used to infect a plant system such as tobacco that is readily accessible to transformation. If explants develop calli on media containing kanamycin, the overexpressed ABC transporter is functionally active in conferring kanamycin tolerance to a plant.

### Example 4

### Cadmium Tolerance

The various binary vectors were also used to test their efficacy in conferring tolerance against cadmium. After transformation, explants were transferred to media containing 500 μM cadmium and, three weeks later, screened for tolerant shoots. This experiment demonstrated that explants infected with the Agrobacterium strain carrying the vector containing Krh1 developed cadmium-tolerant shoots that could be regenerated into whole plants. Almost all explants infected with this strain produced at least one shoot. We also found Krh2, 5, 7, and 8 to provide tolerance to cadmium, if overexpressed. Even higher levels of tolerance can be obtained by operably linking the ABC transporter genes to strong promoters such as the promoter of the potato ubiquitin-7 gene or the 35S promoter of figwort mosaic virus.

The fact that Krh5 provides tolerance whereas Krh4 does not indicates that slight differences in amino acid sequence may be essential for functional activity. The two proteins share 98.4% identity.

### Example 5

### Tolerance Against Deoxynivalenol

Interestingly, the Krh1 gene also provided some tolerance against 45 μM deoxynivalenol (DON). In this case, about half of the explants produced one DON-tolerant shoot. Another gene that provided DON tolerance was Krh5.

### Example 6

### Identification of New Selectable Agents

Apart from kanamycin, cadmium, and DON, it is possible to use any compound that arrests plant cell development as selective agent. ABC transporter genes such as Krh1-8 can be tested for efficacy in conferring tolerance against such a selective agent by taking the following steps:
(1) Tissue culture media are prepared that are the same as standard media for transformation and proliferation except that they contain the selection agent of choice. In fact, a series of agar media is prepared, each of which contains the selection agent at a specific concentration. For instance, glyphosate can be used as selection agent at concentrations of 10 μM, 20 μM, 30 μM, 40 μM, 50 μM, 75 μM, and 100 μM;
(2) Explants of a plant system of choice, such as tobacco, are infected with an Agrobacterium strain;
(3) The infected explants are transferred to the series of agar media, and incubated for about four weeks in growth rooms;
(4) The minimal concentration of the selection agent that prevents any proliferation and/or regeneration is determined. Steps 1-4 define the ‘kill curve’ for a selection agent;
(5) The experiment is repeated but explants are now infected with Agrobacterium strains that carry ABC transporter genes positioned within T-DNA borders, and the infected explants are transferred to media containing the minimum concentration of the selection agent;
(6) Any calli that develop on the infected explants are allowed to regenerate, and are then molecularly analyzed to confirm the presence of the ABC transporter. Steps 5 and 6 identify the gene that confers tolerance against the selection agent;
(7) Explants are infected with the ABC transporter that provides tolerance against the selection agent, and the infected explants are transferred to a series of agar media to determine the optimal concentration of the selection agent. This step optimizes the selection system.

By carrying out experiments like the one described above, we determined that none of the tested ABC transporters provided tolerance against 300 μM copper, 37.5 mg/L cyanamide, 50 mg/L hygromycin, and 300 μM zinc.

### Example 7

### ABC Transporter Genes Operably Linked to Terminators

The terminator region that is operably linked to the kanamycin resistance gene is a sequence that contains the signals for mRNA 3′-end processing. Such a terminator is derived from either a gene or, more preferably, from a sequence that does not represent a gene but intergenic DNA. Examples of such preferred and often more effective terminators include a T-rich sequence from Arabidopsis (SEQ ID NO: 23), a DNA fragment from potato (SEQ ID NO: 24), a DNA fragment from alfalfa (SEQ ID NO: 25), or a DNA fragment from tobacco (SEQ ID NO: 26).

### Example 8

### Strong Promoters Driving ABC Transporter Gene Expression

The efficacy of ABC transporters can be increased by operably linking these genes to strong promoters. One such a promoter is the promoter of the potato ubiquitin-7 gene (SEQ ID NO.: 27), which provides high levels of gene expression in most dicotyledonous plant species. For instance, an expression cassette comprising the Krh8 gene linked to the ubi7 promoter provided more effective tolerance against kanamycin than an expression cassette with the Krh8 gene fused to the 35S promoter of cauliflower mosaic virus. Another strong promoter is the 35S promoter of flgwort mosaic virus (SEQ ID NO.: 28).

TABLE 1
Summary of transformation data.

| | | Explants producing | Explants producing |
|---|---|---|---|
| | | calli that display | calli that display |
| | | resistance against | resistance against |
| | | 100 mg L⁻¹ | 75 mg L⁻¹ |
| Vector | Gene | kanamycin | kanamycin |
| pSIM106OD | Bacterial nptII | + | + |
| pSIM1058 | Arabidopsis | + | + |
| | Atwbc19 | | |
| pSIM1073 | Canola Krh1 | − | − |
| pSIM1074 | Canola Krh2 | − | − |
| pSIM1075 | Canola Krh3 | − | − |
| pSIM1070 | Potato Krh4 | + | + |
| pSIM1071 | Potato Krh5 | + | + |
| pSIM1155 | Tomato Krh6 | + | + |
| pSIM1154 | Tomato Krh7 | + | + |
| pSIM1152 | Tobacco Krh8 | + | + |
| pSIM1177 | Atwbc19-Krh8 | + | ND |
| | chimera | | |

Sequences

SEQ ID NO: 1
ATGCCACGTGTTTCTGCTGAATCCCAAGAAATCTCTCTCGACGGCGGCTGGGAGTCACCAACGCTCGGC
GAACTGCTAAAAGATCTCGAAGACGGTCACCGGAAGAAAGACTCCGGCGAAGATGCTTCGGTTCATCAC
GTATTGGATGTCGCTTCCCCTGAAACAAGACCTGTGCCGTTTCTCTTATCCTTCAACAATCTCTGTTAC
GATGTCAGGGGAAAAGCCGACTCGGTCAAAACTCTACTCAACGATGTTTCCGGCGGGGTTTGCGACGGC
GATGTCCTTGCCGTTCTCGGTGCAAGCGGAGCCGGTAAGTCCACGTTGATCGACGCACTAGCGGGACGT
GTGAGTAGCTTGAGAGGTACGGTAACTCTAAACGGAGAGAAAGTTTTGAAAAGTCAACTCCTAAAAGTG
ATATCAGCATACGTCATGCAAGACGATCCCTTGTTTCCGATGCTCACCGTCAAAGAAACACTAATGTTC
GCTTCAGAGTTTCGTCTTCCGAGAAGCTTGTCCAAGTCCAAGAAAATGGAGCGTGTTGAAGCCCTAATA
GACAAGCTAGGGCTCAGAAACGCGGCGGATACAATAATAGGAGACGAAGGACACCGTGGGGTCTCCGGC
GGAGAGCGGCGGCGCGTGTCGATCGGTGCCGACATCATCCACGACCCCATTGTCTTGTTCCTGGACGAA
CCTACTTCGGGGTTGGACTCCACCAACGCCTTTATGGTGGTGCAAGTTCTTAAGCGTATCGCTCGTAGT
GGCAGTATGGTAATTATGTCGATACATCAACCTAGCGCTCGTATCATAGACTTGCTCGACCGTCTTATC
ATCTTATCTCGCGGCAAGAGTGTATTCAGTGGATATCCGACAAGTCTTCCTCAGTTCTTGTCTGATTTC
GGACATCCAGTCCCGGGGAAAGAGAACATCACAGAGTTTGCACTTGACCTAGTCCGAGAGCTAGAAGGA
TCGACCAAAGGAACCGAAGAGTTAGTAGAGTTCAACGAGAAGTGGCAACAGAACCAATCTCCTCGAGCC
ACGCCAATGACCACTCCTTACAAAGCATTGTCTCTAAAAGAATCCATCACTACAAGTGTTTCCAGAGGC
AAACTAGTCTCCGGCTCGACCAGCTCCAACCCCATCTCAATGGAGACAGTACCATACGCAAACACGCCA
TTGGTCGAGGCATATATATTATCCAAACGTTACATTAAAAACTGGTCCCGCACCCCCGAGCTCATTATA
ACACGGCTCGCTACGGTCCTGGTGACTGGTCTTATCTTAGCTACTATATATTGGAGGCTGGACAACACT
CCACGAGGTGCACAAGAGAGAATGGCTTTCTTTTCATTCGCCATGTCCACAATGTTCTACACCTGTGCA
GACAACCTCCCTGTCTTTATCCATGAACGTTACATTTTCTTGAGAGAGACAACTCACAATGCATACAGG
ACATACTCATACGTTATATCTCACGTTCTCGTGTCTCTGCCTCAGCTACTCGCTCTCTCCATTGCATTT
GCTGTTACCACGTTCTGGACAGTTGGTTTAAGCGGTGGACTAGAGAGCTTCTTGTATTACCGTCTCATT
ATCTACGCAGCCTTTTGGTCTGGTTCCTCTTTCGTTACCTTCATATCCGGTCTTATTCCGAATGTCATG
ATAAGTTTCATGGTCACTATTTCCTATCTTTCGTACTGTCTACTGATGGGTGGATTCTTCATTAACCGT
GATCGGATACCGGGTTACTGGATATGGTTTCATTACATCTCATTGATGAAGTATCCTTATGAAGCTGTC
TCGATCAATGAGTTTGATGACCCATCTCGATGTTTTGTAAGAGGAGTTCAAGTATTTGATGGTACGCTT
TTCGCCAAAGTGCCTGATGCGATAAAGGTTAAGATGTTTGATACACTGGGTAACTCTTTAGGAACTAAG
ATAACGGAGTCCACATGCTTGAGAACAGGGCCTGACTTGCTTTTGCAGCAAGGTATATCTCAGTTGAGC
AAATGGGATTGCTTGTGGGTTACGTTTGCTTGGGGTATCTTCTTTAGGATCTTGTTTTACTTGTCCTTG
TTGTTTGGAAGCAAGAATAAAAGGACGTGA
SEQ ID NO: 2
MPRVSAESQEISLDGGWESPTLGELLKDLEDGHRKKDSGEDASVHHVLDVASPETRPVPFLLSFNNLCY
DVRGKADSVKTLLNDVSGGVCDGDVLAVLGASGAGKSTLIDALAGRVSSLRGTVTLNGEKVLKSQLLKV
ISAYVMQDDPLFPMLTVKETLMFASEFRLPRSLSKSKKMERVEALIDKLGLRNAADTIIGDeghrgvsg
gerrrvslGADIIHDPIVLFLDEPTSGLDSTNAFMVVQVLKRIARSGSMVIMSIHQpSAriidlldrli
ilSRGKSVFSGYPTSLPQFLSDFGHPVPGKENITEFALDLVRELEGSTKGTEELVEFNEKWQQNQSPRA
TPMTTPYKALSLKESITTSVSRGKLVSGSTSSNPISMETVPYANTPLVEAYILSKRYIKNWSRTPELII
TRLATVLVTGLILATIYWRLDNTPRGAQERmaffsfamstmfYTCADNLPVFIHERYIFLRETTHNAYR
TYSYVISHVLVSLPQLLALSIAFAVTTFWTVGLSGGLESFLYYRLIIYAAFWSGSSFVTFISGLIPNVM
ISFMVTISYLSYCLLMGGFFINRDRIPGYWIWFHYISLMKYPYEAVSINEFDDPSRCFVRGVQVFDGTL
FAKVPDAIKVKMFDTLGNSLGTKITESTCLRTGPDLLLQQGISQLSKWDCLWVTFAWGIFFRILFYLSL
LFGSKNKRT
SEQ ID NO: 3
ATGGTGGAGCACGACACTCTCGTCTACTCCAAGAATATCAAAGATACAGTCTCAGAAGACCAAAGGGCT
ATTGAGACTTTTCAACAAAGGGTAATATCGGGAAACCTCCTCGGATTCCATTGCCCAGCTATCTGTCAC
TTCATCAAAAGGACAGTAGAAAAGGAAGGTGGCACCTACAAATGCCATCATTGCGATAAAGGAAAGGCT
ATCGTTCAAGATGCCTCTGCCGACAGTGGTCCCAAAGATGGACCCCCACCCACGAGGAGCATCGTGGAA
AAAGAAGACGTTCCAACCACGTCTTCAAAGCAAGTGGATTGATGTGATAACATGGTGGAGCACGACACT
CTCGTCTACTCCAAGAATATCAAAGATACAGTCTCAGAAGACCAAAGGGCTATTGAGACTTTTCAACAA
AGGGTAATATCGGGAAACCTCCTCGGATTCCATTGCCCAGCTATCTGTCACTTCATCAAAAGGACAGTA
GAAAAGGAAGGTGGCACCTACAAATGCCATCATTGCGATAAAGGAAAGGCTATCGTTCAAGATGCCTCT
GCCGACAGTGGTCCCAAAGATGGACCCCCACCCACGAGGAGCATCGTGGAAAAAGAAGACGTTCCAACC
ACGTCTTCAAAGCAAGTGGATTGATGTGATATCTCCACTGACGTAAGGGATGACGCACAATCCCACTAT
CCTTCGCAAGACCTTCCTCTATATAAGGAAGTTCATTTCATTTGGAGAGGACACGCTGAAATCACCAGT
CTCTCTCTACAAATCTATCTCT
SEQ ID NO: 4
TTGATTTTAATGTTTAGCAAATGTCCTATCAGTTTTCTCTTTTTGTCGAACGGTAATTTAGAGTTTTTT
TTGCTATATGGATTTTCGTTTTTGATGTATGTGACAACCCTCGGGATTGTTGATTTATTTCAAAACTAA
GAGTTTTTGCTTATTGTTCTCGTCTATTTTGGATATCAATCTTAGTTTTATATCTTTTCTAGTTCTCTA
CGTGTTAAATGTTCAACACACTAGCAATTTGGCTGCAGCGTATGGATTATGGAACTATCAAGTCTGTGG
GATCGATAAATATGCTTCTCAGGAATTTGAGATTTTACAGTCTTTATGCTCATTGGGTTGAGTATAATA
TAGTAAAAAAATAGG
SEQ ID NO: 5
ATGCCACGTGTTTCTGCTGAATCCCAAGAAATATCATTCGACGGCGGCAGCGAACCGACGCTCGGAGAG
CTCCTGAAAGATTTCGACGGAGGTGACCGGAAGAAAAACTCCGGCGAAGATGCTTCGACTCATCACATA
CTTGATCTCACATCCCCTGAAATAAGACCCGTACCGTTTCTCTTGTCCTTCAACAACCTCAGCTACGAC
ATCGTACATCGCCGGCGGTTTGACTTCTCTCGAGGAAAGCCAGCTTCAGTGAAAACTCTACTCAACGAT
GTTTCCGGCGAGGCTTGCGACGGAGACATCCTAGCCGTTCTCGGAGCAAGCGGAGCGGGAAAGTCCACG
TTGATCGACGCGCTAGCGGGACGCGTGAGTAGCCTGAGAGGCACGGTAACTCTAAACGGAGAGAAGATC
TTGCAAACTCGTTTGCTGAAAGTGATATCAGCTTACGTCATGCAAGACGATCTTTTGTTCCCGATGCTC
ACCGTCAAAGAAACTCTAATGTTCGCTTCAGAGTTTCGTCTCCCGAGAAGCTTGTCCAAGTCCAAGAAA
ATGGAGCGTGTTCAAACCCTAATAGACCAGTTAGGGCTCAGAAACGCGGCGGATACCATAATAGGAGAC
GAGGGACACCGTGGAGTCTCTGGTGGAGAGCGGAGGCGCGTGTCGATAGGAATCGATATCATCCACGAT
CCTATCCTCTTGTTCCTTGATGAACCTACGTCCGGGTTGGATTCAACCAACGCGTTTATGGTGGTTCAG
GTAAGGCAGCCGGATAAAACATTCCTCTTATTATCTTCAAAATTTTTATATAGTTTACCATATGTTTTA
AAATAAAATTACTTACCTAAATGGCTAAATCTCAGATCCGGTTCGGTTTTCAGGTTCTTAAACGTATAG
CTAGGAGTGGTAGTATCGTAATTATGACAATACACCAACCTAGCGCTCGAGTTCTTGACTTGCTTGATC
GTCTTATCATCTTATCTCGCGGCAAGAATGTTTTCAGCGGTTCTCCGACAAGTCTTCCTCAGTTCTTGT
CTGATTTCGGACATCCTATCCCGGAGAAAGAGAACATAACCGAGTTCGCACTTGACCTAGTTCGTCAGC
TTGAAGGATCTAGTGAAGGAACCAGAGAGTTGGTTAAATTCAACGAAAAGTGGCAACAAAACCAATCTG
CTCGAGCCACGCCAATGACCACACCTTACCAAGCCTTGTCTCTAAAAGAATCCATTACCGCAAGTGTTT
CTAGAGGCAAACTAGTCTCCGGTTCAACCAGTTCCAATCCCATTTCCATGGACTCGGTATCTTCATACG
CAAACCCACCCTTGGTCGAGACCTTCATCTTAGCCAAACGGTACATGAAAAACTGGATCCGGACACCCG
AGCTCTTAGGGACAAGGATCGCCACTGTCATGGTCACTGGTCTTCTCTTAGCTACTATATACTGGAGGC
TTGACAACACTCCACGAGGTGCACAAGAGCGGATGGCTTTCTTTGCATTTGGCATGTCCACGATGTTCT
ACGTCTGTGCAGACAACGTTCCAGTTTTTCTCCAAGAACGGTTCATTTTCTTGAGAGAGACAACGCGCA
ACGCATACAGAACATCTTCGTACGTAATCTCTCACTCTCTTGTCTCTCTGCCTCAGCTACTTGCTCTCT
CAATTGCATTTGCTGCGACCACGTTCTGGACTGTTGGTTTAAGCGGTGGACTAGAGAGCTTCCTTTATT
ACTGCCTCATAATCTACGCAGGCTTTTGGTCTGGATCCTCTTTTGTCACCTTCGTATCCGGTTTGGTTC
CGAATGTCATGATAAGTTTCATGATCACTATTGCCTATCTTTCCTACTGTCTACTCTTGGGTGGATTCT
ACATTAACCGGGATCGGATACCGGTTTACTGGATATGGTTTCATTACATTTCATTGTTGAAGTATCCCT
ACGAAGCTGTCTTAATCAACGAGTTTGATGACCCATCTCGCTGTTTTGTTAGAGGAGTCCAAGTGTTTG
ATGGTACGCTTTTGGCGAAAGTGCCTGATGCGATGAAGGTTAAGCTCCTCGATACACTGAGTAGCTCTT
TAGGAACAACGATAACGGAGTCCACATGCTTGAGAACAGGGCCTGACTTACTTATGCAGCAAGGTATTT
CTCAGTTGAGCAAATGGGATTGTTTGTGGATTACGTTAGCTTGGGGTCTCTTCTTTAGGATCTTGTTTT
ACTTCTCCTTGCTGTTTGGAAGCAAGAATAAAAGGACGTGA
SEQ ID NO: 6
ATGCCACGTGTTTCTGCTGAATCCCAAGAAATCTCATTCGACGGCGGCAACGAACCGACGCTCGGAGAG
CTCCTGAAAGATTTCGACGGAGGTGACCGGAAGAAAAACTCCGGCGAAGATGCTTCGACTCATCACATA
CTTGATCTCACTTCCCCTGAAACAAGACCCGTACCGTTTCTCTTGTCCTTCAACAACCTCAGCTACGAC
ATCGTACATCGCCGGCGGTTTGTCTTCTCTCGACCAAAGCCAGCTTCAGTGAAACCTCTACTCAACGAT
GTTTCCGGCGAGGCTTGCGACGGAGACATCCTAGCCGTTCTCGGAGCAAGCGGAGCCGGAAAGTCCACG
TTGATCGACGCGCTAGCGGGACGCGTGGGTAGCTTGAGAGGCACGGTAACTCTAAACGGAGAGAAGATC
TTGCAAACTCGTTTGCTGAAAGTGATATCAGCTTACGTCATGCAAGACGATCTTTTGTTCCCGATGCTC
ACCGTCAAAGAAACTCTAATGTTCGCTTCAGAGTTTCGTCTCCCGAGAAGCTTGTCCAAGTCCAAGAAA
ATGGAGCGTGTTCAAACCCTAATAGACAAGTTAGGGCTTAGAAACGCGGCGGATACGATAATAGGAGAC
GAAGGTCACCGTGGAGTCTCCGGTGGAGAGCGGCGGCGCGTGTCGATAGGAATCGATATCATCCACGAT
CCTATCCTCTTGTTCCTTGATGAACCTACATCCGGGTTGGATTCAACCAATGCGTTTATGGTTGTGCAG
GTCGGATGAAACATTCGTCTTATCTTCAAAATTTTAAATAGTTACTATATATTTCAATTTTTTTAAATT
AAAATTACTCTCCGAAATCTCAGATCCGGTTCTGTTTTCAGGTTCTTAAACGTATAGCTAGGAGTGGTA
GTATCGTAATTATGACAATACATCAACCTAGCGCTCGAGTCCTTGACTTGCTTGATCGTCTTATCATCT
TATCTCGCGGCGAGAATGTTTTCAGCGGTTCTCCGACAAGTCTTCCTCAGTTCTTGTCTGATTTCGGAC
ATCCTATCCCGGAGAAAGAGAACATAACCGAGTTCGCACTCGACCTAGTACGACAACTCGAAGGGTCCA
GCGAAGGAACCAGAGAGTTAGTTGAGTTCAACGAGAAGTGGCAACAGAACCATTCTGCTCGAGCCACGC
CAATGACCACACCTTACCAAGCCTTGTCTCTAAAAGAATCCATTACCGCAAGTGTTTCGAGAGGCAAGC
TAGTCTCCGGTTCAACCAGTTCCGATCCAATTTCCATGGACTCTGTATCTTCATACGCAAACCCGCCAC
TGGTCGAGACCTTTATCTTAGCCAAACGGTACATGAAAAACTGGATCCGGACACCGGAGCTCATAGGGA
CACGGATCGCCACTGTCATGGTGACTGGTCTTCTCTTAGCTACTATATACTGGAGGCTTGACAACACTC
CGAGAGGTGCACAAGAGAGGATGGCTTTCTTTGCATTTGGTATGTCAACAATGTTCTACGTCTGTGCGG
ACAACGTTCCTGTTTTTCTCCAAGAACGGTTCATTTTCTTGAGGGAGACAACGCGCAACGCATACAGAA
CATCTTCGTACGTAATCTCTCACTCTCTTGTCTCTCTGCCTCAGCTACTTGCTCTCTCAATTGCATTTG
CTGCGACCACGTTCTGGACTGTTGGTTTAAGCGGTGGACTAGAGAGCTTCCTTTATTACTGCCTCATAA
TCTACGCAGGCTTTTGGTCTGGATCCTCTTTTGTCACCTTCGTATCCGGTTTGGTTCCGAATGTCATGA
TAAGTTTCATGATCACTATTGCCTATCTTTCCTACTGTCTACTCTTGGGTGGATTCTACATTAACCGGG
ATCGGATACCGGTTTACTGGATATGGTTTCATTACATTTCATTGTTGAAGTATCCCTACGAAGCTGTCT
TAATCAACGAGTTTGATGACCCATCTCGCTGTTTTGTTAGAGGAGTCCAAGTGTTTGATGGTACGCTTT
TGGCGAAAGTGCCTGATGCGATGAAGGTTAAGCTCCTCGATACACTGAGTAGCTCTTTAGGAACAACGA
TAACGGAGTCCACATGCTTGAGAACAGGGCCTGACTTACTTATGCAGCAAGGTATTTCTCAGTTGAGCA
AATGGGATTGTTTGTGGATTACGTTAGCTTGGGGTCTCTTCTTTAGGATCTTGTTTTACTTCTCCTTGC
TGTTTGGAAGCAAGAATAAAAGACGTGA
SEQ ID NO: 7
ATGTCAAGGATAGTAGCGGAAAATATGTTACAAGGGGGAGAAAATGTACAATTTTATGATCAAAGAGTA
CAACAAGCAATGGAGATGTCACAAGCCAGCGCGTACTCTTCACCCACCCTAGGCCAAATGCTAAAGCGC
GTGGGAGACGTGAGAAAAGAAGTCACCGGCGACGAAACTCCGGTGCACCGGATTCTCGATATGAGTGAT
ACTCAAAGCATATCATCTCACTCTCTTCCTTTTGTACTCTCCTTCAACAACCTCACCTACAGCGTAAAA
GTTCGCCGGAAAATGTCTTTTCCGGCAATACTCCGGCAACCGGCCACCGGAGTTTCCACCGGCGATCCC
GTCGCCGGAGAAAACTTGTTCTCGAACACAAAATTCCTCCTGAACAATATCTCCGGCGAGGCACGGGAC
GGCGAGATAGTCGCCGTCCTGGGTGCATCAGGGTCGGGGAAATCGACCCTGATCGATGCCCTCGCGAAT
AGGATCGCGAAGGAGAGTTTAAAAGGAACGATAACGTTGAACGGAGAGCCACTTGATTCGAGATTATTG
AAAGTAATCTCAGCATATGTAATGCAAGATGATCTTTTATATCCAATGTTGACAGTTGAAGAGACGTTA
ATGTTTGCAGCTGAATTCAGATTGCCACGTACTTTGTCAAAATCAAAAAAGAAAATGAGAGTTCAAGCT
TTGATTGATCAATTAGGACTACGAAATGCTGCAAAAACAATCATTGGTGATGAGGTAAGTTATATATAG
AGTATAATGATGACTACAAACTGATCATATTTATTTTTTTAACATGCATTTAATAAAAATTTACTATTT
TGAACAGGGTCATCGTGGAGTGTCTGGTGGTGAAAGACGACGAGTTTCGATTGGAATTGATATTATTCA
TGACCCTATCATATTGTTTTTAGACGAACCAACTTCAGGTCTTGATTCGACTAGTGCATACATGGTGGT
GAAAGTTCTTCAACGAATTGCTCAAAGTGGAAGTATTGTGATCATGTCAATTCATCAGCCAAGTTATCG
AATTCTCGGGTTATTGGATCGGATGCTCTTCTTGTCCCGTGGTCAAACGGTTTATAGCGGGTCACCTAT
GAACCTCCCACATTTTTTTGCTGATTTTGGTCACCCAATTCCAGATAGTGAAAATCGAACAGAGTTTGC
TCTGGATCTGATTCGGGAACTAGAAGGGTCCCCAGGAGGGACAAAAAGTTTGGTTGAGTTCAACAAAAC
ATGGGAAAATACTAAAAGGAGTAATGAAAATCCTGGAACCCTAACACCTACTCATGGATTGTCATTGAA
AGAAGCAATTAGCGCGAGTATTTCAAGAGGAAAGTTGGTTTCAGGGACAACGAGTGATATTCATACAAG
TCCAGCATCAATGGTTCCAACTTACGCGAATCCATTTTGGATTGAAATGGTTGTCTTGTCCAAGAGGTC
ATTTACAAATTCTTGGAGGGTGCCAGAGTTGTTTGGTATCCGTCTAGGGGCAATCGTGGTAACGGGGTT
CATCCTAGCTACCATGTTTTGGCAACTTGATGATTCCCCTAAAGGGGTTCAAGAAAGGCTTGGTTTCTT
TGCATTTGCTATGTCAACAACTTTCTATACTTGCGCGGACGCGTTGCCTGTGTTCCTCCAAGAGAGGTA
CATTTTCATGAGGGAGACTGCTTATAATGCTTATAGGAGATCTTCCTATTGTCTATCGCATGCTATAGT
TTCTTTGCCAGCATTGATCTTTCTTAGCTTTGCATTTGCCGCTATAACTTTTTGGGCTGTAGGCCTTGT
AGGTGGATTTTCGGGCTTTTTGTTCTATTTCGCAATAATACTAGCCTCCTTTTGGGCCGGGAATTCATT
TGTCACGTTCCTCTCCGGTGTAGTTCCTAGTGTCATGTTAGGTTACACCATTGTGGTCGCGATCCTAGC
CTATTTCCTCCTCTTCTCAGGATTCTTCATCAATCGCGATAGGATTCCACCTTATTGGATATGGTTTCA
CTACCTATCTCTGGTGAAATATCCTTATGAAGCTGTGTTACAAAATGAATTTGATGATGCAACTAAGTG
TTTTGTCAAAGGGATTCAATTGTTTGATAATTCACCACTTGGAAATGTGCCTAATGCATTGAAGGAAAA
ATTGTTGAGTACAATGAGTAACACATTAAATGTCAAAATTACAAGTTCAACATGTGTGACTACTGGGGC
TGATATATTGGTTCAACAAGGGATTACTGATTTAAGTAAGTGGAATTGTTTGTGGATTACTATTGCATG
GGGGTTTTTCTTCAGGGTTTTGTTTTACTTTAGCTTGTTGCTTGGAAGTAAGAACAAGAGAAGGTGA
SEQ ID NO: 8
ATGTCAAGGATAGTAGCGGAAAATATGTTACAAGGGGGAGAAAATGTACAATTTTATAATCAAAGAGTA
CAACAAGCCATGGAGATGTCACAAGCCAGCGCGTACTCTTCACCCACCCTAGGCCAAATGCTAAAGCGC
GTGGGAGACGTGAGAAAGGAAGCCACCGGCGACGAAACTCCGGTGCACCGGATTCTCGATATGAGTGAT
ACTCAAAGCATATCATCTCACTCTCTTCCTTTTGTACTCTCCTTCAACAACCTCACCTACAGCGTAAAA
GTCCGCCGGAAAATGCCTTTTCCAGCGATACTCCGGCGACCGGCCGCCGGAGTTTCCACCGGTGATCCC
ATCGCCGGAGAAAATCTGTTCACGAACACAAAATTCCTCCTGAACAATATCTCCGGCGAGGCCCGGGAC
GGCGAGATAGTCGCCGTCCTGGGTGCATCAGGGTCGGGGAAATCGACCCTGATCGATGCCCTCGCGAAT
AGGATCGCGAAGGAGAGTTTAAAAGGAACGATAACGTTAAACGGAGAGCCACTTGATTCGAGATTGTTG
AAAGTAATCTCAGCATATGTAATGCAAGATGATCTTTTATATCCAATGTTGACAGTTGAAGAAACATTA
ATGTTTGCAGCTGAATTCAGATTGCCACGTACTTCATCAAAATCAAAAAAGAAAATGAGAGTTCAACGT
TTGATTGATCAATTAGGACTACGAAATGCTGCAAAAACAATCATTGGTGATGAGGTAACGTTATATATA
CAGTATAATTTTTCATCGATGCCTACAAACTGATCATTTTTTTTTTAACATTTAATAAAAATTTACTAT
TTTGAACAGGGTCATCGTGGAGTGTCTGGTGGTGAAAGACGACGAGTTTCGATTGGAATTGATATTATT
CATGACCCTATCATATTGTTTTTAGACGAGCCAACTTCAGGTCTTGACTCGACTAGTGCATATATGGTG
GTGAAGGTTCTACAACGAATTGCTCAAAGTGGAAGTATTGTTATCATGTCAATTCATCAGCCAAGTTAT
CGAATTCTCGGGTTATTGGATCGGATGCTCTTCTTGTCCCGTGGTCAAACGGTTTATAGTGGGTCACCT
ATGAACCTCCCACATTTTTTTGCTGATTTTGGTCACCCAATACCGGATAGTGAAAATAGAACAGAGTTT
GCTCTGGATCTAATTCGCGAACTAGAAGGGTCCCCTGGAGGGACAAAAAGTTTGGTTGAGTTCAACAAA
ACATGGGAAAATACTAAAAGGAGTAATGAAAATCCTGAAATCCAAACACCTACTCATGGATTGTCATTG
AAAGAAGCAATTAGCGCGAGTATTTCAAGAGGGAAGTTGGTTTCAGGGACAACGAGTGATATTCATACT
AGTCCAGCATCAATGGTTCCAACTTACGCGAATCCATTTTGGATTGAAATGCTTGTGTTGTCCAAGAGA
TCATTTACGAATTCTTGGAGGGTGCCAGAGTTATTTGGTATTCGTCTAGGGGCAATCGTGGTCACGGGG
TTCATCCTAGCTACCATGTTTTGGCAACTTGATGATTCCCCTAAAGGGGTTCAAGAAAGGCTTGGTTTC
TTTGCATTTGCTATGTCAACAACTTTCTATACTTGCGCGGACGCGTTGCCTGTGTTTCTCCAAGAGAGG
TACATTTTCATGAGGGAGACTGCTTATAATGCTTATAGGAGATCTTCCTATTGTCTATCTCATGCTATA
GTTTCTTTGCCAGCATTGATCTTTCTTAGCTTTGCATTTGCTGCTATAACTTTTTGGGCTGTAGGCCTT
GTAGGTGGATTTTCGGGCTTTTTGTTCTATTTCGCGATAATACTAGCCTCCTTCTGGGCCGGGAATTCA
TTTGTCACGTTCCTCTCCGGTGTAGTTCCTAGTGTCATGTTAGGTTACACCATAGTGGTCGCGATCCTA
GCCTATTTCCTCCTCTTCTCAGGATTCTTCATCAATCGCGATAGGATTCCACCTTATTGGATATGGTTT
CACTACCTGTCTCTGGTGAAATATCCTTATGAAGCTGTGTTACAAAATGAATTTGATGATGCAACAAAG
TGTTTTGTCAAAGGGATTCAATTGTTTGATAATTCACCACTTGGAAATGTGCCTAATGCATTGAAGGAA
AAATTGTTGAGTACAATGAGTAACACATTAAATGTCAAAATTACAAGTTCAACATGTGTGACTACTGGG
GCTGATATATTGGTTCAACAAGGGATTACTGATTTAAGTAAGTGGAATTGTTTGTGGATTACTATTGCA
TGGGGGTTTTTCTTTAGGGTTTTGTTTTACTTTAGCTTGTTGCTTGGAAGTAAGAACAAGAGAAGGTGA
SEQ ID NO: 9
ATGTCAAAAGTTGTAGCTCAAAATGTTTCACCGGTAAGAGATTGTGTACCTCTTTATGATCGGAGACAG
ACGGTAGAAATGTCGTCGCCGACGTTTGCTCAGTTGTTGAACAACGTCGGAGATCACGTCGCCGGCGAC
GAAACTGAAACTCCAGTTCACCATGTTCTGACGATGCAGCCGCAGAACTCTATTCCGTTTGTACTGTCA
TTCAGCAACCTCACATACAGCGTGACAGTTCGCCGGAAAAACATTTTTCCGGCAATGTCTAGGGGCTTG
ACAGAGGAAGCGCCGGTCACGAGGACGAAAGTGCTTTTACATGATATATCAGGGGAGGCGCGTGATGGA
GAGCTACTCGCAGTGCTCGGCGCGTCTGGTTCGGGGAAATCGACGTTGATCGATGCTTTAGCTAATCGA
ATTGCGAAGGAGAGCTTGAAAGGGGAGATAAAATTGAACGGTGAGAAATTGCATACGAAGTTGCTGAAA
GTGATATCGGCGTACGTAATGCAAGACGATCTCCTTTATCCGATGCTTACAGTTGAAGAAACTTTAATG
TTCTCAGCAGAGTTTCGCCTTCCACGAACTCTATCGAAATCGAAGAAGAAAAGTAGAGTTCAAGCATTA
ATCGATCAATTAGGACTCAGAAACGCCGCCAAAACTATAATCGGCGATGAAGGACACCGAGGAGTCTCC
GGCGGTGAAAGACGGAGAGTTTCGATCGGAATCGACATCATCCATGATCCGATCATCTTATTTCTCGAT
GAACCTACTTCCGGTCTCGATTCCACCAATGCTTTCATGGTGGTTAAAGTTCTTCAACGAATCGCACAG
AGCGGGAGTATTGTGATAATGTCGATTCATCAGCCAAGTAATCGAATTCTCGGTTTACTTGATCGATTA
ATCTTCTTATCACGTGGACAAACTGTTTACAGTGATTCACCGTTCAATTTGCCCCAATTTTTCGCTGAT
TTTGGTCAATCAATTCCAGAAAACGAGAACAGAACAGAGTTCGCCCTTGATTTAATCCGCGAACTCGAA
GGCACACCTGATGGAACAAATAATTTAGTTGAATTCAACCGAAAATGGAAAAGTTCTTCAACAACATTG
ACAATATATGATCTATCACTAAAGGAAGCAATAAGTGCAAGCATTTCCAGAGGAAAATTGGTTTCTGGT
GCTGCTAATCCTACCTCTATGGTTCCTACTTTTGCAAATCCAATATGGACCGAAATGGCAGTACTGTCA
AAGCGTTCATTCACAAACTCATGGCGTATGCCGGAGATTTTCTTCGTCCGTTTCAGTGCAGTAATGGTC
ACGGGTTTTATCCTCTCAACCATTTTCTGGCGGCTCGACAATTCCCCTAAAGGCATATGGGAACGCCTT
GGGTTTATAGCATTCGCAGTGTCAACAACTTACTATATATGCGCGGAGGCGTTGCCCGTTTTCATTCAC
GATAGGTACATTTTCATGAGGGAAACAGCTTACAATGCTTATCGGAGATCATCATATTGCCTCTCTCAC
GCTTTGGTTTCGCTACCATCATTGATAATCCTCTCTCTATCGTTCGCAGCCCTGACTTTCTGGGCTGTT
GGCCTAGACGGTGGAGCTTCGGGCTTTTTGTTCTACTTCTCTGTCGTTCTAGCTTCAATCTGGGCGGGG
AATTCATTCGTCACTTTCCTCTCTAGTGTCGTTCCTCATGTCATGATCGGTTACACAATCGTAGTTGCA
TTACTCGGATACTTCCTCCTCTTCAGTGGATTCTACATGAATCGCGATAGGATCCCATCTTACTGGATA
TGGTTTCATTACCTCTCCCTCGTGAAATACCCATATGAAGCAGTGTTACTGAACGAATTCAAAGACCCC
ACGAAATGCTTCGTTCGTGGGGTTCAATTGTTCGATAACAGCCAATTGGGGGATTTACCGAATTCAATA
AAGGAGAAATTATTGGACAATATGAGCGAAACGTTGAACGCAACGATAACAAGTTCCACTTGTTTAACG
AGTGGCGCAGATGTATTGATGCAACAAGGGATAACTCAACTGAACAAATGGAATTGTTTGTGGGTAACA
ATTGCATGGGGATTTTTCTATAGGATTTTGTTTTATTTCACTTTGTTATTGGGAAGTAAAAATAAAAGG
AGGTAA
SEQ ID NO: 10
ATGAGTAGAATTGTTGCTCAAAATGTATCACCGGTAAGAGATTGCGTACCTCTTTATGATCGGAGACAA
ATGGTAGAAATGTCATCGCCGACGTTTGCTCAGTTGTTGAACAACGTCGGAGATGATGTCACCGGCGAC
GAAACTGGAGCTTCAGTTCACCGAGTTCTGACAATGGAACTGCCTCTGCAGCAATCGATTCCATTTGTA
TTGTCATTCAGTAACCTCACATACAGCGTGAGAGTCCGCCGGAAAAATATTTTTCCGGCGATGTCCGGC
CGCCGGAACCGGACAGATGAACCGCGGTGCACGAGGACGAAGGTGCTTTTGAATGATATATCAGGAGAG
GCGCGCGACGGAGAACTACTCGCGGTGCTCGGCGCGTCTGGTTCGGGGAAATCGACGTTGATCGATGCT
TTAGCTAATCGAATTTCGAAAGATAGCTTGAAAGGGGAGATGAAATTGAACGGTGAGCCATTGCATTCG
AAGTTGCTGAAAGTGATATCGGCGTACGTAATGCAAGACGATCTCCTTTATCCGATGCTTACAGTTGAA
GAAACTTTAATGTTCTCAGCAGAGTTTCGCCTTCCACGAACTCTATCGAAATCGAAGAAGAAGAGTAGA
GTTCAAGCATTAATCGATCAATTAGGACTCCGAAACGCCGCCAAAACTATAATCGGCGATGAAGGCCAC
CGAGGAGTCTCCGGCGGTGAAAGACGGAGAGTTTCGATCGGAATCGACATCATCCATGATCCGATCATC
TTATTCCTCGATGAACCTACCTCCGGTCTCGATTCCACCAGTGCTTTTATGGTGGTTAAAGTTCTTCAA
CGAATCGCACAGAGCGGAAGTATTGTGATAATGTCGATTCATCAACCAAGTTATCGAATTCTCGGTTTA
CTTGATCGATTAATCTTCTTATCACGCGGACAGACTGTTTACAGTGGTTCACCGTTCAATTTGCCCCAA
TTTTTCGCTGATTTTGGTCATCCAATTCCAGAAAACGAGAATAAAACAGAGTTCGCGCTTGATTTAATC
CGCGAACTCGAAGGGTCACCTAATGGAACAAGTAGTTTAGTTGAATTCAATCGAAAATGGAAAAATTCT
TCAACAACATCGACAATATATGATCTATCACTAAAGGAAGCAATAAGTGCAAGCATTTCCAGAGGAAAA
TTGGTTTCTGGTGCTGCTAATCCTACGTCTATGGTTCCTACTTTTGCAAATCCAATATGGACCGAAATG
GCAGTACTATCAAACCGATCATTCACAAACTCATGGCGTATGCCGGAGATTTTCGCTGTACGTTTCGGT
GCAGTAATGGTCACGGGTTTCATCCTCGCCACCATGTTCTGGCGGCTCGATGATTCCCCTAGAGGTGTA
AGGGAACGGATTGGGTTTTTCGCGTTTGCAATGTCTACAACTTACTATACATGCGCGGAGGCATTGCCC
GTTTTCATTCACGAGAGGTTCATTTTCATGAGGGAAACGGCTTACAATGCTTATCGGAGATCATCATAT
TGCCTCTCTCACGCTTTGGTTTCGATACCATCATTGGTATTCCTCTCTCTATCGTTCGCAGCTCTGACT
TTCTGGGCTGTTGGCCTAGACGGTGGAGCTTCGAGCTTTCTCTTCTATTTATCTGTCGTCCTTGCTTCC
TTTTGGGCTGGTAACTCATTCGTCACGTTCCTCTCTGGTGTCATTCCTCATGTCATGATTGGATACGTA
ATCGTTGTAGCAATTTTCGCGTATTACCTTCTCTTCAGTGGTTTCTTCTTGAATCGTGATAGGATTCCG
TCTTACTGGATATGGTTTCATTACATCTCGCTCGTGAAATACCCATATGAAGCAGTGTTGCAGAACGAA
TTCAAAGATCCCATGAAATGCTTCGTTCGTGGGATTCAATTGTTCGATAACAGCCCACTCGGGGATGTT
CCGATTTCGTTGAAGGAGAAATTGTTGGACAGTATAAGCAATACGTTGAACGTAAGGATAACAAGTTCG
ACATGTGTGGTGACTGGTGCAGATATATTGGTGCAACAAGGGATAACTCAACTGAACAAATGGAATTGC
TTGTGGGTAACAATTGCATGGGGATTTTTCTTTAGGATTTTGTTTTATTTCACTTTGTTATTGGGAAGT
AAAAATAAAAGGAGGTAA
SEQ ID NO: 11
ATGTCAAGAATAATTAATGCATCACCAGTTAGAGATAGCATACCACTTTATGATCGGAGACAAGTTGTA
GAAATGTCATCGCCGACGTTTGGTCAGTTGTTGAAGAACGTCGGCGATGTCACCGGCGACGACGAAAGT
CCACTTCATCAAGCTCTTACCATGGACCCGCATCACTCTAATATTCCCTTTGTACTCGCATTCAACAAC
CTCACATACAGTGTGAAAGTCCGCCGGAAGGTCAATTTTCCGGCGATCTCACGTAGCCGGAGCAGCCGC
AGCCCCGCTGAGGAAATCCCGTCCACCAGAACAAAGGTGCTTTTGAATGACATCTGTGGAGAAGCGCGT
GATGGAGAGCTACTCGCGGTTTTAGGGGCGTCTGGTTCGGGAAAATCGACGCTCATTGATGCGTTAGCG
AATCGTATTGCGAAAGATAGCTTGAAAGGGACAGTAACACTGAACGGCGAGCCACTGCACTCGAAATTA
CTCAAAGTCATATCGGCTTACGTAATGCAAGACGATCTTCTCTACCCAATGCTTACAGTAGAAGAAACG
TTAATGTTCGCAGCTGAGTTTAGGCTTCCACGGAGTCTATCAAAATCGAAGAAAAAATCCAGAGTTCAA
GCTTTAATCGATCAATTAGGGCTCAGAAACGCCGCGAAAACTATCATCGGCGATGAAGGTCACCGTGGT
GTCTCCGGCGGTGAACGGCGGCGCGTTTCAATCGGAATTGACATAATCCATGACCCGATTATTCTCTTT
CTCGACGAGCCAACTTCGGGTCTCGATTCCACCAGTGCTTTTATGGTGATTAAAGTACTTCAACGAATC
GCGCAGAGTGGCAGTATTGTAATTATGTCAATCCATCAGCCGAGTTACAGAATTGTCGGTTTACTTGAC
CGGTTGATTTTCCTATCACGTGGACAAACTGTTTACAGTGGCTCGCCGTTGAATTTGCCACAATTTTTT
GCTGATTTTGGAAATCCAATACCTGAAAATGAGAACCGCATAGAGTTCGCGCTTGATTTAATTCGCGAA
CTCGAAGGGTCAGGTAGGACAAGGAGCTTAGTCGATTTCAACAAAACATGGCAACATATGAAACGGACT
AGTAGTACAAATCAGAATACTGAAACAACAGGTAGAAATAGAAATCGTTTATCGTTAAAGGAAGCGATA
AGTGCTAGTATTTCTAGAGGAAAATTGGTTCCTGGTTCAACACATGTTGCTACTAGTCCTACTTCTATG
GTTCCTACTTTTGCAAATCCAATATGGACAGAAATAGCTGTACTTTCAAAGCGATCATTCACTAACTCG
TGGCGCATGCCCGAGATTTTTGCTGTTCGATTTGGTGCGGTTATGGTTACGGGGTTTATACTGGCTACT
ATGTTTTGGCGACTTGACAGTTCACCTAAAGGTGTACAAGAACGGCTTGGATTCTTCGCGTTCGCGATG
TCAACGACTTACTATACATGTGCGGACGCATTGCCCGTTTTTATTCAAGAAAGGTACATTTTCATGAGG
GAAACGGCTTATAATGGATATAGAAGATCATCTTATTGTCTTTCTCATGCTTTGACTTCGATACCAGCG
TTGATTTTCCTCGCTCTGTCATTCGCCGCCGTGACATTCTGGGCTGTTGGCTTAGATGGTGGATTTTCT
AGCTTTTTGTTCTATTTCACTGTCATTTTGGCTTCGTTTTGGGCAGGGAATTCATTCGTTACGTTCCTT
TCTGGTGTCGTGCCTCATGTCATGCTCGGATACACAATCGTGGTAGCAATTTTAGCCTACTTCCTACTC
TTTAGTGGATTCTTCATGAATCGTGATAGAATCCCGTCTTACTGGATCTGGTTCCATTATATTTCGCTA
GTGAAATACCCGTATGAAGCTGTGTTACAGAACGAATTTGATGATCCCACGAAATGCTTCGTTCGTGGG
ATTCAAATGTTCGATAACAGTCCACTTGGGGCTGTTCCGAATTCGTTAAAGGAGAAGTTGTTGAGCAGT
ATTAGCAGTACATTGAATATGAGGATTACAAGTTCAACATGTGTGACTACTGGATCAGATATATTGGTG
CAACAAGGGATTACACAATTGAGCAAGTGGAATTGCCTTTGGGTAACTATTGCATGGGGGTTTTTGTTT
AGGATTTTGTTTTATTTCTGCTTGTTGCTTGGAAGTAAGAATAAGAGAAGTTAA
SEQ ID NO: 12
MPRVSAESQEISFDGGSEPTLGELLKDFDGGDRKKNSGEDASTHHILDLTSPEIRPVPFLLSFNNLSYD
IVHRRRFDFSRGKPASVKTLLNDVSGEACDGDILAVLGASGAGKSTLIDALAGRVSSLRGTVTLNGEKI
LQTRLLKVISAYVMQDDLLFPMLTVKETLMFASEFRLPRSLSKSKKMERVQTLIDQLGLRNAADTIIGD
eghrgvsggerrrvsigiDIIHDPILLFLDEPTSGLDSTNAFMVVQVLKRIARSGSIVIMTIHQPSArv
ldlldrliilSRGKNVFSGSPTSLPQFLSDFGHPIPEKENITEFALDLVRQLEGSSEGTRELVKFNEKW
QQNQSARATPMTTPYQALSLKESITASVSRGKLVSGSTSSNPISMDSVSSYANPPLVETFILAKRYMKN
WIRTPELLGTRIATVMVTGLLLATIYWRLDNTPRGAQERMAFFAFGMSTMFYVCADNVPVFLQERFIFL
RETTRNAYRTSSYVISHSLVSLPQLLALSIAFAATTFWTVGLSGGLESFLYYCLIIYAGFWSGSSFVTF
VSGLVPNVMISFMITIAYLSYCLLLGGFYINRDRIPVYWIWFHYISLLKYPYEAVLINEFDDPSRCFVR
GVQVFDGTLLAKVPDAMKVKlldtlssslgttitestCLRTGPDLLMQQGISQLSKWDCLWITLAWGLF
FRILFYFSLLFGSKNKR
SEQ ID NO: 13
MPRVSAESQEISFDGGNEPTLGELLKDFDGGDRKKNSGEDASTHHILDLTSPETRPVPFLLSFNNLSYD
IVHRRRFVFSRGKPASVKPLLNDVSGEACDGDILAVLGASGAGKSTLIDALAGRVGSLRGTVTLNGEKI
LQTRLLKVISAYVMQDDLLFPMLTVKETLMFASEFRLPRSLSKSKKMERVQTLIDKLGLRNAADTIIGD
eghrgvsggerrrvsigiDIIHDPILLFLDEPTSGLDSTNAFMVVQVLKRIARSGSIVIMTIHQPSArv
ldlldrliilSRGENVFSGSPTSLPQFLSDFGHPIPEKENITEFALDLVRQLEGSSEGTRELVEFNEKW
QQNHSARATPMTTPYQALSLKESITASVSRGKLVsgstssdpismdsvssYANPPLVETFILAKRYMKN
WIRTPELIGTRIATVMVTGLLLATIYWRLDNTPRGAQERMAFFAFGMSTMFYVCADNVPVFLQERFIFL
RETTRNAYRTSSYVISHSLVSLPQLLALSIAFAATTFWTVGLSGGLESFLYYCLIIYAGFWSGSSFVTF
VSGLVPNVMISFMITIAYLSYCLLLGGFYINRDRIPVYWIWFHYISLLKYPYEAVLINEFDDPSRCFVR
GVQVFDGTLLAKVPDAMKVKlldtlssslgttitestCLRTGPDLLMQQGISQLSKWDCLWITLAWGLF
FRILFYFSLLFGSKNKR
SEQ ID NO: 14
MSRIVAENMLQGGENVQFYDQRVQQAMEMSQASAYSSPTLGQMLKRVGDVRKEVTGDETPVHRILDMSD
TQSISSHSLPFVLSFNNLTYSVKVRRKMSFPAILRQPATGVSTGDPVAGENLFSNTKFLLNNISGEARD
GEIVAVLGASGSGKSTLIDALANRIAKESLKGTITLNGEPLDSRLLKVISAYVMQDDLLYPMLTVEETL
MFAAEFRLPRTLSKSKKKMRVQALIDQLGLRNAAKTIIGDEGHRGVSGGERRRVSIGIDIIHDPIILFL
DEPTSGLDSTSAYMVVKVLQRIAQSGSIVIMSIHQPSYRILGLLDRMLFLSRGQTVYSGSPMNLPHFFA
DFGHPIPDSENRTEFALDLIRELEGSPGGTKSLVEFNKTWENTKRSNENPGTLTPTHGLSLKEAISASI
SRGKLVSGTTSDIHTSPASMVPTYANPFWIEMVVLSKRSFTNSWRVPELFGIRLGAIVVTGFILATMFW
QLDDSPKGVQERLGFFAFAMSTTFYTCADALPVFLQERYIFMRETAYNAYRRSSYCLSHAIVSLPalif
lsfafaaitfWAvglvggfsgflfyfAIILASFWAGNSFVTFLSGVVPSVMLGYTIVVAILAYFLLFSG
FFINRDRIPPYWIWFHYLSLVKYPYEAVLQNEFDDATKCFVKGIQLFDNSPLGNVPNALKEKLLSTMSN
TLNVKITSSTCVTTGADILVQQGITDLSKWNCLWITIAWGFFFRVLFYFSLLLGSKNKRR
SEQ ID NO: 15
MSRIVAENMLQGGENVQFYNQRVQQAMEMSQASAYSSPTLGQMLKRVGDVRKEATGDETPVHRILDMSD
TQSISSHSLPFVLSFNNLTYSVKVRRKMPFPAILRRPAAGVSTGDPIAGEnlftntkfllnnISGEARD
GEIVAVLGASGSGKSTLIDALANRIAKESLKGTITLNGEPLDSRLLKVISAYVMQDDLLYPMLTVEETL
MFAAEFRLPRTSSKSKKKMRVQALIDQLGLRNAAKTIIGDEGHrgvsggerrrvsigidiihdpiiLFL
DEPTSGLDSTSAYMVVKVLQRIAQSGSIVIMSIHQPSYRILGLLDRMLFLSRGQTVYSGSPMNLPHFFA
DFGHPIPDSENRTEFALDLIRELEGSPGGTKSLVEFNKTWENTKRSNENPEIQTPTHGLSLKEAISASI
SRGKLVSGTTSDIHTSPASMVPTYANPFWIEMLVLSKRSFTNSWRVPELFGIRLGAIVVTGFILATMFW
QLDDSPKGVQERLGFFAFAMSTTFYTCADALPVFLQERYIFMRETAYNAYRRSSYCLSHAIVSLPalif
lsfafaaitfWAvglvggfsgflfyfAIILASFWAGNSFVTFLSGVVPSVMLGYTIVVAILAYFLLFSG
FFINRDRIPPYWIWFHYLSLVKYPYEAVLQNEFDDATKCFVKGIQLFDNSPLGNVPNALKEKLLSTMSN
TLNVKITSSTCVTTGADILVQQGITDLSKWNCLWITIAWGFFFRVLFYFSLLLGSKNKR
SEQ ID NO: 16
MSKVVAQNVSPVRDCVPLYDRRQTVEMSSPTFAQLLNNVGDHVAGDETETPVHHVLTMQPQNSIPFVLS
FSNLTYSVTVRRKNIFPAMSRGLTEEAPVTRTKVLLHDISGEARDGELLAVLGASGSGKSTLIDALANR
IAKESLKGEIKLNGEKLHTKLLKVISAYVMQDDLLYPMLTVEETLMFSAEFRLPRTLSKSKKKSRVQAL
IDQLGLRNAAKTIIGDeghrgvsggerrrvsigidiihdpiiLFLDEPTSGLDSTNAFMVVKVLQRIAQ
SGSIVIMSIHQPSNRILGLLDRLIFLSRGQTVYSDSPFNLPQFFADFGQSIPENENRTEFALDLIRELE
GTPDGTNNLVEFNRKWKSSSTTLTIYDLSLKEAISASISRGKLVSGAANPTSMVPTFANPIWTEMAVLS
KRSFTNSWRMPEIFFVRFSAVMVTGFILSTIFWRLDNSPKGIWERLGFIAFAVSTTYYICAEALPVFIH
DRYIFMRETAYNAYRRSSYCLSHalvslpsliilslsfaalTFWAVGLDGGASGFLFYFSVVLASIWAG
NSFVTFLSSVVPHVMIGYTIVVAllgyfllfsgfyMNRDRIPSYWIWFHYLSLVKYPYEAVLLNEFKDP
TKCFVRGVQLFDNSQLGDLPNSIKEKLLDNMSETLNATITSSTCLTSGADVLMQQGITQLNKWNCLWVT
IAWGFFYRILFYFTLLLGSKNKR
SEQ ID NO: 17
MSRIVAQNVSPVRDCVPLYDRRQMVEMSSPTFAQLLNNVGDDVTGDETGASVHRVLTMELPLQQSIPFV
LSFSNLTYSVRVRRKNIFPAMSGRRNRTDEPRCTRTKVLLNDISGEARDGELLAVLGASGSGKSTLIDA
LANRISKDSLKGEMKLNGEPLHSKLLKVISAYVMQDDLLYPMLTVEETLMFSAEFRLPRTLSKSKKKSR
VQALlDQLGLRNAAKTIIGDeghrgvsggerrrvsigidiihdpiiLFLDEPTSGLDSTSAFMVVKVLQ
RIAQSGSIVIMSIHQPSYRILGLLDRLIFLSRGQTVYSGSPFNLPQFFADFGHPIPENENKTEFALDLI
RELEGSPNGTSSLVEFNRKWKNSSTTSTIYDLSLKEAISASISRGKLVSGAANPTSMVPTFANPIWTEM
AVLSNRSFTNSWRMPEIFAVRFGAVMVTGFILATMFWRLDDSPRGVRERIGFFAFAMSTTYYTCAEALP
VFIHERFIFMRETAYNAYRRSSYCLSHALVSIPslvflslsfaaltfwaVGLDGGASSFLFYLSVVLAS
FWAGNSFVTFLSGVIPHVMIGYVIVVAIFAYYLLFSGFFLNRDRIPSYWIWFHYISLVKYPYEAVLQNE
FKDPMKCFVRGIQLFDNSPLGDVPISLKEKLLDSISNTLNVRITSSTCVVTGADILVQQGITQLNKWNC
LWVTIAWGFFFRILFYFTLLLGSKNKR
SEQ ID NO: 18
MSRIINASPVRDSIPLYDRRQVVEMSSPTFGQLLKNVGDVTGDDESPLHQALTMDPHHSNIPFVLAFNN
LTYSVKVRRKVNFPAISRSRSSRSPAEEIPSTRTKVLLNDICGEARDGELLAVLGASGSGKSTLIDALA
NRIAKDSLKGTVTLNGEPLHSKLLKVISAYVMQDDLLYPMLTVEETLMFAAEFRLPRSLSKSKKKSRVQ
ALIDQLGLRNAAKTIIGDEGHRGVSGGERRRVSIGIDIIHDPIILFLDEPTSGLDSTSAFMVIKVLQRI
AQSGSIVIMSIHQPSYRIVGLLDRLIFLSRGQTVYSGSPLNLPQFFADFGNPIPENENRIEFALDLIRE
LEGSGRTRSLVDFNKTWQHMKRTSSTNQNTETTGRNRNRLSLKEAISASISRGKLVPGSTHVATSPTSM
VPTFANPIWTEIAVLSKRSFTNSWRMPEIFAVRFGAVMVTGFILATMFWRLDSSPKGVQERLGFFAFAM
STTYYTCADALPVFIQERYIFMRETAYNGYRRSSYCLSHALTSIPALIFLALSFAAVTFWAVGLDGGFS
SFLFYFTVILASFWAGNSFVTFLSGVVPHVMLGYTIVVAILAYFLLFSGFFMNRDRIPSYWIWFHYISL
VKYPYEAVLQNEFDDPTKCFVRGIQMFDNSPLGAVPNSLKEKLLSSISSTLNMRITSSTCVTTGSDILV
QQGITQLSKWNCLWVTIAWGFLFRILFYFCLLLGSKNKRS
SEQ ID NO: 19
ATGAATCTATCACTCAGCGGTAGAAAGATTGCCATGACACGTGTTTCGGCGGAAACTCAGTATATCACT
CCCATCGGATCACCAACCCTCGACGAGTTGCTGAAAGACTGCGACAGTTTCCGAAAAGGAGATTCCGGC
GACGGCGTAAAAAGCGACGATCCTGCACATCACATAATAGACGTCGAAGCCTTGTACGTAAAACCTGTC
CCGTACGTCTTAAACTTTAACAATCTTCAATACGATGTCACACTTCGCCGGCGGTTTGGCTTCTCACGG
CAAAACGGAGTAAAGACTCTACTCGATGATGTTTCCGGAGAGGCTTCTGACGGCGAGCTCCTCGCGGTT
TTAGGGGCGTCTGGTTCGGGAAAATCGACGCTCATTGATGCGTTAGCGAATCGTATTGCGAAAGATAGC
TTGAAAGGGACAGTAACACTGAACGGCGAGCCACTGCACTCGAAATTACTCAAAGTCATATCGGCTTAC
GTAATGCAAGACGATCTTCTCTACCCAATGCTTACAGTAGAAGAAACGTTAATGTTCGCAGCTGAGTTT
AGGCTTCCACGGAGTCTATCAAAATCGAAGAAAAAATCCAGAGTTCAAGCTTTAATCGATCAATTAGGG
CTCAGAAACGCCGCGAAAACTATCATCGGCGATGAAGGTCACCGTGGTGTCTCCGGCGGTGAACGGCGG
CGCGTTTCAATCGGAATTGACATAATCCATGACCCGATTATTCTCTTTCTCGACGAGCCAACTTCGGGT
CTCGATTCCACCAGTGCTTTTATGGTGATTAAAGTACTTCAACGAATCGCGCAGAGTGGCAGTATTGTA
ATTATGTCAATCCATCAGCCGAGTTACAGAATTGTCGGTTTACTTGACCGGTTGATTTTCCTATCACGT
GGACAAACTGTTTACAGTGGCTCGCCGTTGAATTTGCCACAATTTTTTGCTGATTTTGGAAATCCAATA
CCTGAAAATGAGAACCGCATAGAGTTCGCGCTTGATTTAATTCGCGAACTCGAAGGGTCAGGTAGGACA
AGGAGCTTAGTCGATTTCAACAAAACATGGCAACATATGAAACGGACTAGTAGTACAAATCAGAATACT
GAAACAACAGGTAGAAATAGAAATCGTTTATCGTTAAAGGAAGCGATAAGTGCTAGTATTTCTAGAGGA
AAATTGGTTCCTGGTTCAACACATGTTGCTACTAGTCCTACTTCTATGGTTCCTACTTTTGCAAATCCA
ATATGGACAGAAATAGCTGTACTTTCAAAGCGATCATTCACTAACTCGTGGCGCATGCCCGAGATTTTT
GCTGTTCGATTTGGTGCGGTTATGGTTACGGGGTTTATACTGGCTACTATGTTTTGGCGACTTGACAGT
TCACCTAAAGGTGTACAAGAACGGCTTGGATTCTTCGCGTTCGCGATGTCAACGACTTACTATACATGT
GCGGACGCATTGCCCGTTTTTATTCAAGAAAGGTACATTTTCATGAGGGAAACGGCTTATAATGGATAT
AGAAGATCATCTTATTGTCTTTCTCATGCTTTGACTTCGATACCAGCGTTGATTTTCCTCGCTCTGTCA
TTCGCCGCCGTGACATTCTGGGCTGTTGGCTTAGATGGTGGATTTTCTAGCTTTTTGTTCTATTTCACT
GTCATTTTGGCTTCGTTTTGGGCAGGGAATTCATTCGTTACGTTCCTTTCTGGTGTCGTGCCTCATGTC
ATGCTCGGATACACAATCGTGGTAGCAATTTTAGCCTACTTCCTACTCTTTAGTGGATTCTTCATGAAT
CGTGATAGAATCCCGTCTTACTGGATCTGGTTCCATTATATTTCGCTAGTGAAATACCCGTATGAAGCT
GTGTTACAGAACGAATTTGATGATCCCACGAAATGCTTCGTTCGTGGGATTCAAATGTTCGATAACAGT
CCACTTGGGGCTGTTCCGAATTCGTTAAAGGAGAAGTTGTTGAGCAGTATTAGCAGTACATTGAATATG
AGGATTACAAGTTCAACATGTGTGACTACTGGATCAGATATATTGGTGCAACAAGGGATTACACAATTG
AGCAAGTGGAATTGCCTTTGGGTAACTATTGCATGGGGGTTTTTGTTTAGGATTTTGTTTTATTTCTGC
TTGTTGCTTGGAAGTAAGAATAAGAGAAGTTAA
SEQ ID NO: 20
MNLSLSGRKIAMTRVSAETQYITPIGSPTLDELLKDCDSFRKGDSGDGVKSDDPAHHIIDVEALYVKPV
PYVLNFNNLQYDVTLRRRFGFSRQNGVKTLLDDVSGEASDGELLAVLGASGSGKSTLIDALANRIAKDS
LKGTVTLNGEPLHSKLLKVISAYVMQDDLLYPMLTVEETLMFAAEFRLPRSLSKSKKKSRVQALIDQLG
LRNAAKTIIGDEGHRGVSGGERRRVSIGIDIIHDPIILFLDEPTSGLDSTSAFMVIKVLQRIAQSGSIV
IMSIHQPSYRIVGLLDRLIFLSRGQTVYSGSPLNLPQFFADFGNPIPENENRIEFALDLIRELEGSGRT
RSLVDFNKTWQHMKRTSSTNQNTETTGRNRNRLSLKEAISASISRGKLVPGSTHVATSPTSMVPTFANP
IWTEIAVLSKRSFTNSWRMPEIFAVRFGAVMVTGFILATMFWRLDSSPKGVQERLGFFAFAMSTTYYTC
ADALPVFIQERYIFMRETAYNGYRRSSYCLSHALTSIPALIFLALSFAAVTFWAVGLDGGFSSFLFYFT
VILASFWAGNSFVTFLSGVVPHVMLGYTIVVAILAYFLLFSGFFMNRDRIPSYWIWFHYISLVKYPYEA
VLQNEFDDPTKCFVRGIQMFDNSPLGAVPNSLKEKLLSSISSTLNMRITSSTCVTTGSDILVQQGITQL
SKWNCLWVTIAWGFLFRILFYFCLLLGSKNKRS
SEQ ID NO: 21
MATSAHTVLDVDSGGGAATAAAGPPVPYLLSFTDLSYSVRKGGGGVLSCLPSSRRRRHSNRLASADAPA
P
PDAPTKALLDGISGEARDGELFAVMGASGSGKSTLVDALAGRIARESLRGAVELNGEPLHGRRLRAISA
Y
VMQDDLLYPMLTVRETLLFAAEFRLPRALSPDKKRARVDALIDQLGLARAADTIIGDEAHRGVSGGERR
R
VSIGTDIVHDPILLFLDEPTSGLDSASAFMVVQVLRRIAQSGSVVIMTIHQPSARILNILDRLLLLSRG
R
TVYAGTPVGLKPFFSEFGDPIPDNENPAEFALDTIRELEHQPDGAAPLADFNVKWQSMHAALPAADSKD
S
KRCTMPLELAITESVSRGKLVAGSGSGTASSTSVPTFANPLSVEVWVLMKRSFTNTGRMPELFVMRLGT
I
MVTGFILATIFWRLDDTPKGVQERLGFFAMANSTMFYVCADALPVFVQERHIYLRETAHNAYRRLSYVF
A
NAVVAFPPLVFLSLAFAVTTFFAVGLAGGGGSFLFFVLIILASFWAGSGFVTFLSAVVPHVMLGYTVVV
A
ILAYFLLFSGFFINRDRIPSYWIWFHYLSLVKYPYQAVLQNEFRDATRCFSRGVEMFDGTPIGAMSRAV
K
LKVLDAISKTLGTNMTANTCVTTGADVLAQQAVTDIGKWKCLLVTVAWGFFFRALFYVVLLVGSKNKRR
SEQ ID NO: 22
MARIVAANDDDSMELNTISSIHDSTLGQLLKNVSDVRKMAIGDETPVHESLNQDYNDGYMRTVPFVLSF
D
NLTYNVSVRPKLDFRNLFPRRRTEDPEIAQTARPKTKTLLNNISGETRDGEIMAVLGASGSGKSTLIDA
L
ANRIAKGSLKGTVKLNGETLQSRMLKVISAYVMQDDLLFPMLTVEETLMFAAEFRLPRSLPKSKKKLRV
Q
ALIDQLGIRNAAKTIIGDEGHRGISGGERRRVSIGIDIIHDPILLFLDEPTSGLDSTSAFMVVKVLKRI
A
QSGSIVIMSIHQPSHRVLGLLDRLIFLSRGHTVYSGSPASLPRFFTEFGSPIPENENRTEFALDLIREL
E
GSAGGTRGLIEFNKKWQEMKKQSNRQPPLTPPSSPYPNLTLKEAIAASISRGKLVSGGESVAHGGATTN
T
TTLAVPAFANPMWIEIKTLSKRSMLNSRRQPELFGIRIASVVITGFILATVFWRLDNSPKGVQERLGFF
A
FAMSTMFYTCADALPVFLQERYIFMRETAYNAYRRSSYVLSHAIVSFPSLIFLSVAFAATTYWAVGLDG
G
LTGLLFYCLIILASFWSGSSFVTFLSGVVPSVMLGYTIVVAILAYFLLFSGFFINRNRIPDYWIWFHYM
S
LVKYPYEAVLQNEFSDATKCFVRGVQIFDNTPLGELPEVMKLKLLGTVSKSLGVTISSTTCLTTGSDIL
R
QQGVVQLSKWNCLFITVAFGFFFRILFYFTLLLGSKNKRR
SEQ ID NO: 23
ATCCACCACTCGTTTTAACATCCTGATACCTCCCGNCGGCGCCAATTAAAAATTATATTTATATANATA
T
TCACTTTATTATATATTTTATTTTTATAAATAAATTTTATATTTTAAATATTTATTCTTACATTATATT
A
AATTTATTTTTTTACATAAAATATAATTATTTTTTAAATTATTAAATATTATATTTATTTTTTATTTTT
A
TTTATATTTTTATAATTATATAATTCATTTTTTTTTTTTTCTATTTTATTTATAATTTTATTTTACTCA
T
AATTATTTTTATTTATTAATTTTTTTTTATATATTTTTTTATTTTTTATTATTATTTTATTTTTATAAT
T
ATTATCATTATATTTTTTTATTTTAAATTATTTTTATATTTTATTTTTATTATATTTATTTCTAAATTT
T
TTATTTTTATTTATTTTTTTATTTAAAATTTATTATAATTTTATTTCATTTCTTTTATTATT
SEQ ID NO: 24
ATTACACTCGAGGGACACGTATATTTTTTATTGTAATATAGAGAAAAAGAGTGAGGTGTCGAGTTTTGA
GAAGGGTTACGGTGGTGATTTGTTATGGGATGTCACGTGGGAAAGTTTGAGATGCAGCCGGCGTGGGCC
CCGTGAAGTAGAGCAATTTGATTGGATGTTTTTGCAATTTGAATGTTTATTTTTGGAGTTTTATGGGAG
TTTGATTGTAACCTCTTCTTTTTCTTTGATATTTTCTTTCATGATCTTTTCTTTTGCCTTTGATATTTT
CTTGCAATAGTACTTTACTCTTAACTCTGGTTAACTGAATTAATTTGTCTCAATTTATTTGGTTAACAT
AAGAATTTTGGTAC
SEQ ID NO: 25
TGCAGGGCTTTCTCTCAAGATGATTAATTAATTAATTAGTCTCTTCTCTTTGTATGTGTTTGAAGGGAA
TCAAAGTGTAGACTCACAAAGTGTGATTTTTTATAGAATGGATGATAAAGATGTGGATTGGTTTTTGTG
TGGCTCAGATGTGTGGAAGAATGTGTGATTTAGATAATGTAGATGTGCCACAGAGTGCGTGGGCTTAAA
GTGGGTTATCTCATCAGGATTGTTGTGATTGGTTGGATTGGATGTTATCCTCCTACGTGGCTTAGATTT
TTTAGTAAATATACCGTTTTTTTTTTTTTTTTGTTGGAGGGATTAATATACCGTTATTTCTCTTATTTG
ATCTATGAAATATAAAAATGAGTTAAATGACTCTATGGGGCAATATATAGGAAAAGATCAAATGTCAAA
TTAAATTTGACACCAAATTCTAATCATTAATACAACTTTAATCCAACTCTCTCATCAATTCATTAAATA
ATCACATGTTTAATCAAATGACTCAACTTTTAATTTTAAAAATAATTTATTCTTTCTTTTTAATGACTT
GCCAAAAATTCTCTAATGGTACATCTCTCTAATGCGTCATCCTTATTTATTTACAGCAATTAACCCTTA
TTTGTGTAGCTTTTGTCAAATTCCTCAATGACATTTGTACAACCATTTTGGTACAACTTTTGTACAACT
TTCTCTCTCATACTCACATTATGTTTTTATTCTCCCTCTTCCTTTTTCTCTCTCTCTCTCTATTGTTTT
TGACCAATGAAAAGAGAGAACAACAAAATTTTCTCAAATGTTGTAACAAAATTGTTGTCAAAATATCAC
TACTCTTATTCTTATCATCCATATTTCTTTACTCTAATGTTTAATAATGATTTATAACAGTAGTAGGTG
AATACTTTTGTCCAAATAAAAATGTGCACACTTGCTAACTTAAACTTTTATGATTTTTGAGTTATTTGA
CAACATATAAATATATTTTTTTAATAATTTCTTTCTCCACCTACCTAACTGGTC
SEQ ID NO: 26
GGATCCTTTTGGGAGTTATTTATGCTCCCGTTTGGCCATTGATTTTGGCTACTATTTTTCAAGTTAAAT
TCTTTTTTCAACTTCCCAAAAATTGATTTATGACATTTTTTGGATAAAAGTTTTTTTCCACCTACAAAA
TTTAACTTCTTTTTTTCAAATAAAATGCATGTCCAAACACAACTTCAACTTTCAAATATATTTTTTAAC
ATAACTTCAAAAACTCTTTTTTCAAGTTTTAATTATACATATGTTCAACTATGTATTCATTTCTAGTTA
TGTTTATCACGCATTTCATAAGTGAATTTCATACTTATCTTCATGCAAACATATATACTATAAAAGATA
TATTATTCCTAAATACAACATGTGATACGAGATCATTACATTGCAACTGACCTTATTATTTTTAAATTT
TGGACTTCACCAAAAATAGTTGGGTTTTTTAATCGATTTGATTTAATTTTTCGGTTTGGTGCGGTTTTC
CGATTTGGTTT
SEQ ID NO: 27
TCGAGCACATTGATTGAGTTTTATATGCAATATAGTAATAATAATAATATTTCTTATAAAGCAAGAGGT
CAATTTTTTTTTATTATACCAACGTCACTAAATTATATTTGATAATGTAAAACAATTCAATTTTACTTA
AATATCATGAAATAAACTATTTTTATAACCAAATTACTAAATTTTTCCAATAAAAAAAAGTCATTAAGA
AGACATAAAATAAATTTGAGTAAAAAGAGTGAAGTCGACTGACTTTTTTTTTTTTATCATAAGAAAATA
AATTATTAACTTTAACCTAATAAAACACTAATATAATTTCATGGAATCTAATACTTACCTCTTAGAAAT
AAGAAAAAGTGTTTCTAATAGACCCTCAATTTACATTAAATATTTTCAATCAAATTTAAATAACAAATA
TCAATATGAGGTCAATAACAATATCAAAATAATATGAAAAAAGAGCAATACATAATATAAGAAAGAAGA
TTTAAGTGCGATTATCAAGGTAGTATTATATCCTAATTTGCTAATATTTAAACTCTTATATTTAAGGTC
ATGTTCATGATAAACTTGAAATGCGCTATATTAGAGCATATATTAAAATAAAAAAATACCTAAAATAAA
ATTAAGTTATTTTTAGTATATATTTTTTTACATGACCTACATTTTTCTGGGTTTTTCTAAAGGAGCGTG
TAAGTGTCGACCTCATTCTCCTAATTTTCCCCACCACATAAAAATTAAAAAGGAAAGGTAGCTTTTGCG
TGTTGTTTTGGTACACTACACCTCATTATTACACGTGTCCTCATATAATTGGTTAACCCTATGAGGCGG
TTTCGTCTAGAGTCGGCCATGCCATCTATAAAATGAAGCTTTCTGCACCTCATTTTTTTCATCTTCTAT
CTGATTTCTATTATAATTTCTCTCAATTGCCTTCAAATTTCTCTTTAAGGTTAGAAATCTTCTCTATTT
TTGGTTTTTGTCTGTTTAGATTCTCGAATTAGCTAATCAGGTGCTGTTATAGCCCTTA
SEQ ID NO: 28
ATTTAGCAGCATTCCAGATTGGGTTCAATCAACAAGGTACGAGCCATATCACTTTATTCAAATTGGTAT
CGCCAAAACCAAGAAGGAACTCCCATCCTCAAAGGTTTGTAAGGAAGAATTCTCAGTCCAAAGCCTCAA
CAAGGTCAGGGTACAGAGTCTCCAAACCATTAGCCAAAAGCTACAGGAGATCAATGAAGAATCTTCAAT
CAAAGTAAACTACTGTTCCAGCACATGCATCATGGTCAGTAAGTTTCAGAAAAAGACATCCACCGAAGA
CTTAAAGTTAGTGGGCATCTTTGAAAGTAATCTTGTCAACATCGAGCAGCTGGCTTGTGGGGACCAGAC
AAAAAAGGAATGGTGCAGAATTGTTAGGCGCACCTACCAAAAGCATCTTTGCCTTTATTGCAAAGATAA
AGCAGATTCCTCTAGTACAAGTGGGGAACAAAATAACGTGGAAAAGAGCTGTCCTGACAGCCCACTCAC
TAATGCGTATGACGAACGCAGTGACGACCACAAAAGAATTCCCTCTATATAAGAAGGCATTCATTCCCA
TTTGAAGGATCATCAGATACTCAACCAAT

What is claimed is:

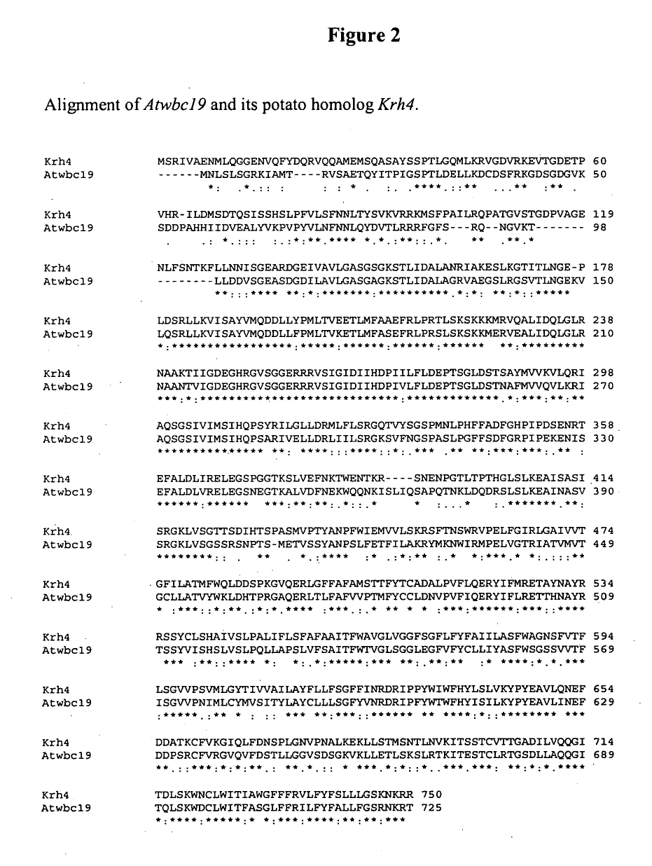

1. An ABC transporter gene, wherein the ABC transporter gene (i) does not comprise the sequence of the Atwbc19 gene depicted in FIG. 1 or FIG. 2, but (ii) confers tolerance to a plant, when it is expressed in the plant, to a selection agent.

2. The ABC transporter gene of claim 1, wherein the encoded ABC transporter comprises the motif A[K/E][E/G]S and the selection agent is kanamycin.

3. The ABC transporter gene of claim 2, wherein the gene encodes a protein that shares at least 80% sequence identity with the protein encoded by a gene selected from the group consisting of SEQ ID NOs: 7-11.

4. The ABC transporter gene of claim 1, wherein the gene encodes a protein that shares at least 80% sequence identity with the protein encoded by a gene selected from the group consisting of SEQ ID NOs: 1, 8, 10, and 11, and wherein the selection agent is cadmium.

5. The ABC transporter gene of claim 1, wherein the gene encodes a protein that shares at least 80% sequence identity with the protein encoded by a gene selected from the group consisting of SEQ ID NOs: 1 and 8, and wherein the selection agent is deoxynivalenol.

6. A method for designing a transformation selection system, comprising (i) producing a kill curve for a selection agent, (ii) identifying an ABC transporter that provides tolerance against the selection agent, and (iii) optimizing the selection system.

7. The method of claim 6, wherein the selection agent is a toxin and selected from the group consisting of kanamycin, neomycin, paramomycin, geneticin, ampicillin, hygromycin, spectinomycin, streptomycin, glyphosate, chlorosulfuron, phosphinothricin, cadmium, zinc, copper, lead, aluminum, or iron;.

8. The method of claim 6, wherein the selection agent is a combination of at least two toxins.

9. A plant comprising a gene that encodes a protein that shares at least 80% sequence identity with the protein encoded by a gene selected from the group consisting of SEQ ID NOs: 1 and 5-11, wherein the gene is operably linked to a foreign promoter and wherein at least one cell of that plant displays tolerance against at least one toxin.
